(19) United States
(12) Patent Application Publication
(10) Pub. No.: US 2004/0132047 A1
(43) Pub. Date: Jul. 8, 2004

(54) Methods for detection of genetic alterations associated with cancer

(76) Inventors: Paolo Fortina, Philadelphia, PA (US); John M. Maris, Moorestown, NJ (US); Craig A. Gelfand, Jackson, NJ (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/606,133

(22) Filed: Jun. 25, 2003

Related U.S. Application Data
(60) Provisional application No. 60/391,515, filed on Jun. 25, 2002.

(51) Int. Cl. C12Q 1/68

(52) U.S. Cl. 435/6

(57) ABSTRACT

Methods are provided for assessing the presence or absence of genetic alterations in a defined polynucleotide region as a means to diagnose and manage malignant disease.

### PRIORITY CLAIM

This application claims the benefit of U.S. Provisional Application 60/391,515 filed Jun. 25, 2002, the entire disclosure of which is incorporated by reference herein.

### GRANT STATEMENT

Pursuant to 35 U.S.C. Section 202(c), it is acknowledged that the United States Government has certain rights in the invention described herein, which was made in part with funds from the National Institutes of Health, Grant No. CA83220.

### FIELD OF THE INVENTION

This invention relates to the fields of genetics, molecular biology and oncology. More specifically, the invention provides methods for assessing the presence or absence of genetic alterations (e.g., single nucleotide polymorphisms) in a defined polynucleotide region as a means to diagnose and manage malignant disease.

### BACKGROUND OF THE INVENTION

Several publications and patent documents are referenced in this application by full citations or by numerals in parentheses in order to more fully describe the state of the art to which this invention pertains. Full citations for these references are found at the end of the specification. The disclosure of these publications and patents is incorporated by reference herein.

Recent scientific and technological advances have accelerated the elucidation of molecular and genetic defects which cause cancer. However, the ability to explain the molecular role of somatic mutations in malignancies has been limited by the lack of rapid and reliable assays for screening large numbers of patient samples.

To date, genetic alterations suspected of causing cancer, such as polymorphisms or mutations in DNA sequences, are most commonly detected by hybridization techniques utilizing allele-specific oligonucleotide (ASO) probes. ASO probes are designed to form either a perfectly matched hybrid or to contain a mismatched base pair at the site of the variable nucleotide residues. Oligonucleotides with 3′ ends complementary to sites of variable nucleotides have been used as ASO primers. The identification of the variable nucleotides is based on the mismatches at the 3′ end which inhibit polymerization reactions. A similar approach is used in oligonucleotide ligation assays, in which two adjacent oligonucleotides are ligated only if there is a perfect match at the termini of the oligonucleotides.

Cleavage of DNA sequences with restriction enzymes is also utilized to identify genetic variations, provided that the variable nucleotide alters (e.g., creates or destroys) a specific restriction site.

Unfortunately, these methods are relatively complex procedures and there are many drawbacks which makes them difficult to use in routine diagnostics. The use of allele specific oligonucleotide probes requires careful optimization of the reaction conditions separately for each application. Fractionation by gel electrophoresis is also required in several of the methods described above. As a result, such methods are not easily automated or used for large throughput screening assays.

There are many factors impeding progress in understanding how tumor-specific genetic alterations influence tumor invasiveness and metastasis. Paramount among them are limitations in the quantity of patient-derived biological reagents and relatively cumbersome techniques necessary to detect salient changes. Furthermore, LOH is determined following radiolabeled PCR, size separation of alleles by gel electrophoresis and autoradiographic analysis. Although widely used, this technique is cumbersome and interpretation of results are often inexact.

The ability to array thousands of cDNA, large insert genomic DNA clones or pre-synthesized oligonucleotides on a glass slide is revolutionizing molecular diagnostics. Microarray technology has mainly been used in cancer research to detect differential gene expression, but also may be used to detect copy number differences in genomic DNA samples or specific gene mutations. These technologies will play an important role in future molecular diagnostic and clinical correlative studies of human cancers associated with somatic mutations.

Clearly a need exists for the development of a rapid, parallel and cost-effective approach to mapping allelic deletion location and size for elucidating the molecular mechanisms underlying the genetic changes associated with malignancy and metastasis.

### SUMMARY OF THE INVENTION

In accordance with the present invention, a novel detection method has been devised for determining the presence or absence of at least one genetic alteration in a target nucleic acid for the diagnosis and management of malignant disease. The inventive method comprises providing a target nucleic acid from a patient sample having a predetermined sequence in the normal population and assessing the target nucleic acid for the extent of loss of heterozygosity relative to predetermined loci, an increase in loss of heterozygosity being correlated with enhanced tumor invasiveness and metastasis.

The target nucleic acid may be assessed by a number of different methods including restriction enzyme mapping, hybridization with allele specific probes, oligomer ligation, DNA sequencing and quantitative PCR.

In an exemplary embodiment, the method of the invention may be used to advantage to identify genetic alterations in the 1p36.3 region of chromosome 1 wherein increased loss of heterozygosity is associated with increased metastasis and poor prognosis in patients with neuroblastoma.

The method for determining the presence or absence of at least one specific nucleotide in a target nucleic acid for the diagnosis and management of malignant disease comprises: (a) providing a detectable amount of a target nucleic acid polymer isolated from a chromosomal region known to be associated with malignancy; (b) hybridizing a detectable amount of the nucleic acid polymer with one or more oligonucleotide primers, each primer having a nucleotide sequence that is complementary to a sequence in the target nucleic acid polymer, such that when the primer is hybridized to the target nucleic acid polymer, the 3′ end of the primer binds to a nucleotide flanking the specific nucleotide at the defined site in the target nucleic acid; (c) exposing the hybridized nucleic acid polymer to a polymerization agent in a mixture containing one or more chain terminating nucleotide triphosphate analogues, at least one of which is detectably labeled, with such label possibly including the intrinsic mass of the nucleotide itself, such that a detectable primer extension product is formed if the labeled nucleotide is complementary to the specific nucleotide at the defined site; (d) analyzing the polymerization mixture of step (c) for the presence or absence of the primer extension product containing the labeled nucleotide at the 3′ end thereof, wherein the identity of the specific nucleotide at the defined site is determined; and (e) assessing the target nucleic acid for loss of heterozygosity in at least one single nucleotide loci, the degree of loss of heterozygosity being correlatable with increased tumor invasiveness and poor patient prognosis.

In a particularly preferred embodiment, the method described above is used to identify genetic alterations associated with neuroblastoma whereby the target nucleic acid of step (e) is compared to the chromosomal region of step (b) which lacks genetic alterations at the 1p36.3 region of chromosome 1, an increase in loss of heterozygosity being associated with increased metastasis and poor prognosis of neuroblastoma.

### BRIEF DESCRIPTION OF THE DRAWINGS

FIG. 1 shows a table listing the chromosomal regions and the genetic alterations associated with neuroblastoma. Four control regions are also provided.

FIG. 2 shows four panels illustrating loss of heterozygosity (LOH) analysis using conventional PCR-based genotyping at simple tandem repeat polymorphic (STRP) loci located at the distal short arm of chromosome 1 (1p36). The four panels show data from four separate tumor (T) and blood (B) paired DNA samples at two different STRP loci, D1S468 and D1S2660.

FIG. 3 is a schematic diagram of an exemplary method of the invention. PCR amplicons are generated containing the SNP site(s) of interest. PCR products are cleaned using any appropriate method. The SNP identifying step follows, in which a specifically designed primer hybridizes to one strand of the amplicon, with the 3′ end of the primer hybridizing to the base just 5′ of the SNP allele. A polymerase and a mixture of terminating nucleotide triphosphates, or functional analogs, is added, with the polymerase catalyzing single-base primer extension of the primer such that the added base is the Watson-Crick complement of the SNP allele. At least two of the terminating nucleotide triphosphates generally bear detectable labels at the two bases appropriate for the SNP being genotyped.

FIG. 4 is a schematic diagram depicting “tag array” as the sorting mechanism for genotyping read-out. The 5′ end of the primers used are complementary to so-called tag sequences. By hybridization, the tags on the primers find their complement tags, facilitating read-out, in particular when the previous biochemical genotyping step has been performed in multiplex. These tag sequences are inert for the biochemical steps, and only become active and relevant in this hybridization step. The location of any particular tag sequence facilitates analysis, such that multiplex reactions are spatially sorted into singlex for read-out. One method of singlex read-out is to use a physical array, such that the tag complements are oriented in a two-dimensional pattern, typical of “DNA chips”. The position of each tag sequence is known by design, such that the physical position after hybridization of each SNP is known, and imaging of the DNA chip, often by fluorescence microscopy, results in the genotype data.

FIG. 5 depicts an exemplary means for determining which SNPs are present or absent in a patient sample based on incorporation of flourescent tagged labels into the PCR amplification product complementary to the SNP location. The Affymetrix GenFlex™ chip, which contains 2000 unique tag sequences can be read using a single hybridization step in which as many as 2000 uniquely tagged primers are present. The number of tags that can be used is limited only by the ability to produce the arrays themselves, of for microparticle arrays, by the number of uniquely identifiable particles that can be produced.

FIG. 6 shows a view of the Orchid Biosciences software which may be utilized in the method of the invention. The graphical user interface shown is a matter of convenience and utility for the user, and does not have any particular bearing on the assay itself.

FIGS. 7A and 7B show the results obtained following performance of the method on a neuroblastoma cell line, LAN-5, and a matched control, LAN-L (lymphocyte-derived DNA from the patient from whom the LAN-5 neuroblastoma cell line was established). Each fluorescence signal in shown as a colored symbol, with intensity of signal depicted on a log scale. The Y-axis shows relative fluorescence (RF) intensity and the X-axis the “percentage value” (PV) of fluorescence at 530 and 570 nm wavelengths. SNPs with RF<2.0 were considered “failed”.

FIGS. 8A and 8B show the results obtained following performance of the method on another neuroblastoma cell line, KCN, and a matched control, KCL.

FIGS. 9A and 9B show the results obtained following performance of the method on the neuroblastoma cell line, KCN, and the matched control, KCL, at the LOH control region, 16 p12-13 (the site of a hereditary neuroblastoma predisposition locus).

FIGS. 10A and 10B show the results obtained following performance of the method on the tumor-(FIG. 10A) and blood-derived (FIG. 10B) genomic DNA samples for one patient (CHOP 341).

FIGS. 11A-11F show the SNP and PCR primers (SEQ ID NOS: 1-282) used to amplify genomic DNA corresponding to known SNPs located in predetermined regions of chromosome 1.

FIGS. 12A and 12B list the SNPs located in the 1p and 16p regions of chromosomes 1 and 16, respectively that were examined for genetic alterations associated with neuroblastoma. The full length SNP nucleic acid sequences may be obtained from the National Institutes of Health SNP database which is accessible at www.ncbi.nlm.nih.gov. Type the “rs” numbers provided in FIGS. 12A and 12B in the search bar of the database to obtain full length sequences.

### DETAILED DESCRIPTION OF THE INVENTION

In accordance with the present invention, a single nucleotide extension (SNE) method has been devised to identify and characterize genetic alterations (e.g., single nucleotide polymorphisms (SNPs)) in tumor samples. The present inventors have determined that the presence or absence of these genetic alterations can be correlated with clinical outcome in patients with cancer. Thus, data obtained using the method of the invention provides the clinician with important information facilitating the diagnosis and management of malignancy. Refined risk assessment and treatment strategies may also be developed from these genetic patterns to help increase patient survival.

A number of cancers are caused by somatic mutations in tumor suppressor genes. One such cancer is neuroblastoma, which is the most common extracranial pediatric solid tumor and the most common cancer of any type diagnosed during infancy. Significant progress has been made in elucidating the genetic basis of neuroblastoma. Amplification of the MYCN proto-oncogene occurs in approximately 20% of primary neuroblastomas and is strongly associated with the presence of metastatic disease.

Other genetic abnormalities associated with neuroblastoma have been described. Brodeur et al. first recognized that deletions of the short arm of chromosome 1 (1p) were a common karyotypic feature of advanced neuroblastomas. Molecular genetic studies have confirmed these observations by documenting loss of heterozygosity (LOH) in 20-35% of primary tumors. Detailed deletion mapping studies have also confirmed the existence of a common region of deletion within chromosome sub-band 1p36.3. It is hypothesized that a single tumor suppressor gene maps within the smallest region of overlap of all deletions at 1p36.3 and that this gene is inactivated in at least one-third of primary neuroblastomas. Several investigators have also documented a strong correlation of 1p LOH with high-risk clinical and biological prognostic variables indicating that 1p allelic deletion occurs in the more malignant subset of neuroblastomas.

Allelic deletion at 11q23 in MYCN single-copy neuroblastomas have also been noted in approximately 15-20% of reported neuroblastoma karyotypes. Constitutional rearrangements of 11q have been observed in some neuroblastoma patients, suggesting that disruption of an 11q gene may predispose to the development of neuroblastoma.

LOH for 11q was detected in 5-32% of primary neuroblastomas using restriction fragment polymorphism markers. Recently, comparative genomic hybridization studies have detected loss of 11q material in 10-31% of neuroblastomas studied.

Deletion of the long arm of chromosome 14 is also a common abnormality in neuroblastomas and is inversely correlated with MYCN amplification. LOH for 14q has been reported in up to 27% of primary neuroblastomas. LOH was highly correlated with allelic deletion at 11q23 and inversely correlated with MYCN amplification. Taken together, allelic deletion at chromosome bands 11q23 and 14q32 may define a unique subset of human neuroblastomas that have an aggressive clinical behavior in the absence of MYCN amplification.

In an exemplary embodiment, the array-based SNE assay of the invention may be used to assess the location and degree of SNPs in genes associated with oncogenesis in patients with cancer. The array-based SNE assay may be used for example to assess different chromosomal regions (e.g., 1p36, 11q23 and 14q32) in pediatric patients with neuroblastoma. Gain or loss of genetic material at each locus correlates with tumor aggressiveness and patient outcome. Thus, screening for the presence and number of SNPs at these loci will greatly enhance the clinician's ability to predict the aggressiveness of the disease and to improve treatment strategies designed for more aggressively growing tumors.

In further embodiments of the invention, the array-based SNE assay may be used to advantage for rapid high-throughput screening of a variety of other human tumor specimens that are known to be associated with somatic mutations, including but without limitation, adenocarcinomas, breast cancer, colorectal cancer, leukemias, lymphomas, ovarian cancer, pancreatic cancer, prostate cancer and retinoblastoma.

The array-based SNE assay provides several advantages over existing genetic screening methods. For example, numerous sequential experiments may be performed in one array-based SNE assay, thereby providing significant cost savings both in terms of reagents and time. Additionally, the amount of DNA required to perform the method is quite small. Finally, the method provides enhanced sensitivity and detects loss of SNPs in the presence of “contaminating” normal DNA.

In an alternative embodiment of the invention, the degree and characterization of LOH can be determined using non SNP-based methods. For example, hybridization probes may be developed that hybridize to the nucleic acid at specific chromosomal locations. The hybridization signals would be approximately half of the intensity as compared to control samples in regions of actual LOH. In addition, quantitative PCR methods may be utilized.

The following description sets forth the general procedures involved in practicing the present invention. To the extent that specific materials are mentioned, it is merely for purposes of illustration and is not intended to limit the invention. Unless otherwise specified, general biochemical and molecular biological procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (1997) (hereinafter “Ausubel et al.”) are used.

I. Definitions:

The following definitions are provided to facilitate an understanding of the present invention:

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989):

Tₘ=81.5° C.+16.6Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “specific binding pair” as used herein includes antigen-antibody, receptor-hormone, receptor-ligand, agonist-antagonist, lectin-carbohydrate, nucleic acid (RNA or DNA) hybridizing sequences, Fc receptor or mouse IgG-protein A, avidin-biotin, streptavidin-biotin, amine-reactive agent-amine conjugated molecule and thiol-gold interactions. Various other determinant-specific binding substance combinations are contemplated for use in practicing the methods of this invention, such as will be apparent to those skilled in the art.

The phrase “detectably labeled” is used herein to refer to any substance whose detection or measurement, either directly or indirectly, by physical or chemical means, is indicative of the presence of the target bioentity in the test sample. Representative examples of useful detectable labels, include, but are not limited to the following: molecules or ions directly or indirectly detectable based on light absorbance, fluorescence, reflectance, light scatter, phosphorescence, or luminescence properties; molecules or ions detectable by their radioactive properties; molecules or ions detectable by their nuclear magnetic resonance or paramagnetic properties. Included among the group of molecules indirectly detectable based on light absorbance or fluorescence, for example, are various enzymes which cause appropriate substrates to convert, e.g., from non-light absorbing to light absorbing molecules, or from non-fluorescent to fluorescent molecules. Additionally, the intrinsic mass of an unlabeled base is considered a detectable feature, as each of the four natural bases has a different mass, making identification by mass, e.g. with a mass spectrometer, a viable detection method.

The phrase “consisting essentially of” when referring to a particular nucleotide means a sequence having the properties of a given SEQ ID NO.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. Chemical tag moieties include such molecules as biotin, which may be added to either nucleic acids or proteins and facilitates isolation or detection by interaction with avidin reagents, and the like. Numerous other tag moieties are known too, and can be envisioned by the trained artisan, and are contemplated to be within the scope of this definition.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

“Loss of Heterozygosity” as used herein refers to the loss of a wild type allele in a tumor DNA. For example, a non-cancerous cell may be heterozygous at a particular locus. However, in a cancer cell, one of the two alleles may be lost or deleted at the particular locus.

“Target nucleic acid” as used herein refers to a previously defined region of a nucleic acid present in a complex nucleic acid mixture wherein the defined wild-tpye region contains at least one known nucleotide variation which may or may not be associated with malignancy.

The term “solid matrix” as used herein refers to any format, such as beads, microparticles, the surface of a microtitration well or a test tube, a dipstick or a filter. The material of the matrix may be polystyrene, cellulose, latex, nitrocellulose, nylon, polyacrylamide, dextran or agarose.

The term “polymerizing agent” as used herein refers to any enzyme which is capable of primer dependent elongation of nucleic acids. Suitable enzymes include, without limitation, T7 DNA polymerase, T4 DNA polymerase, the Klenow fragment of Escherichia coli DNA polymerase and other suitable DNA polymerases, reverse transcriptase and polymerases from thermophilic microbes such as Thermus aguaticus and Thermus thermoohilus.

The term “genetic alteration” as used herein refers to a change from the wild-type or reference sequence of one or more nucleic acid molecules. Genetic alterations include without limitation, base pair substitutions, additions and deletions of at least one nucleotide from a nucleic acid molecule of known sequence.

II. Single Nucleotide Extension Assay:

An exemplary method according to the invention is based on combined SNP-IT™ (Orchid BioSciences) and GenFlex™ (Affymetrix) tag array technologies. Single nucleotide extension (SNE) reactions can be performed following the methods described in U.S. Pat. Nos. 6,004,744 and 6,013,431, the entire disclosures of both being incorporated by reference herein. The reaction products are then hybridized to tag arrays to identify those single nucleotide polymorphisms which are present or absent in a patient sample. The prognosis of the patient is then determined based on the degree of loss of heterozygosity (LOH) at particular SNP loci associated with malignancy.

Single base extension (SNE) is a technique that allows the detection of single nucleotide polymorphisms (SNPs) by hybridizing a single strand DNA probe to a DNA target. The technique is generally applicable to detection of any single base mutation. Briefly, this method first hybridizes a primer to a target sequence suspected of containing a known single nucleotide polymorphism. The primed DNA is then subjected to conditions in which a DNA polymerase adds a labeled dNTP, typically a ddNTP, acyclic nucleotide triphosphate, or any nucleotide capable of being incorporated by a polymerase but chemically incapable of supporting further polymerase activity, if the next base in the template is complementary to the labeled nucleotide in the reaction mixture. Only when the correct base is available in the reaction will a base be incorporated at the 3′-end of the primer. Chain elongation terminates upon the addition of the ddNTP or functional analog.

In a preferred embodiment of the invention, the target DNA can be any human, animal, plant cell or microbe. Most preferably the target DNA is isolated from a human by methods generally known to those of ordinary skill in the art.

In another embodiment of the invention, the SNE reactions are performed by multiplex PCR whereby multiple PCR reactions are performed using various primer sets, preferably 12 to 48 primer sets, in the same sample pool. Ideal primer sets for use in the inventive method are generated from genomic DNA that are known to contain SNPs in defined chromosomal regions of interest. SNPs located in defined chromosomal regions are identified from various SNP databases, including without limitation, the Orchid Biosciences database, NCBI's dbSNP database, The SNP consortium database and the Celera database.

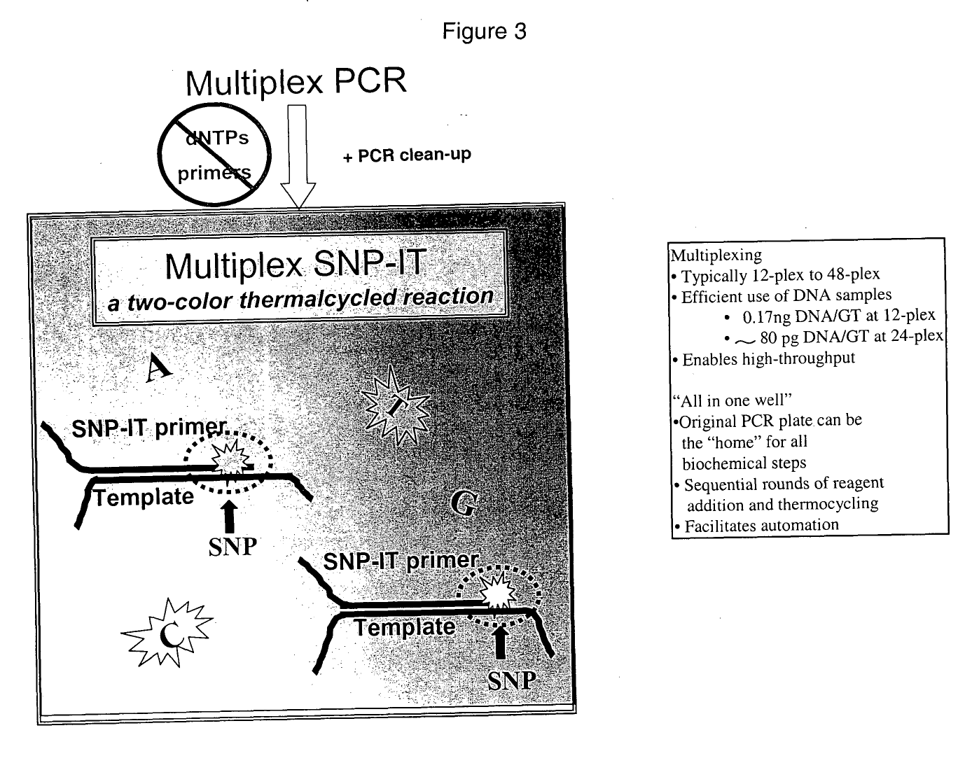

The SNP detection genotyping method described in U.S. Pat. Nos.: 6,004,744 and 6,013,431 is preferred for the purposes of this disclosure, but any SNP genotyping method could be used. A schematic of this method is provided in FIG. 3. Multiplexing, as is possible with this method provides enhanced throughput appropriate for LOH detection, and also provides a key reagent and DNA savings, which is of particular benefit for diagnostic assays where tissue sample may be available only in small quantities. The key to the LOH diagnostic approach is the ability to test enough SNPs to give sufficient LOH information about as many chromosomal sites as necessary. Mutliplexing and array-based assays meet these criteria, but singlex and/or extremely high-throughput assays also fit these basic criteria. With this extension to virtually any genotyping assays, SNP genotyping could be accomplished by any assay, including single-nucleotide extensions using mass spectrometry as the read-out, oligonucleotide ligation assay (OLA), mismatch cleavage methods (e.g. “cleavage assays”), allele-specific hybridization assay (ASH), allele-specific PCR reactions (such as ARMS), other enzymatic methods common in the art, such as restriction enzymes (restriction fragment length polymorphism (RFLP)), and also, potentially, non-SNP polymorphisms, such as microsatellites, small tandem repeats (STRs). One important criterion is the physical density of polymorphisms (e.g. number of polymorphisms as a function of position on the chromosome) that can be probed. SNPs are the most-dense possible markers, allowing LOH maps to have finer resolution that can pinpoint individual genes in the LOH region. STR maps will not have such dense resolution, but can still provide adequate information about larger regions that are involved in the LOH genotype.

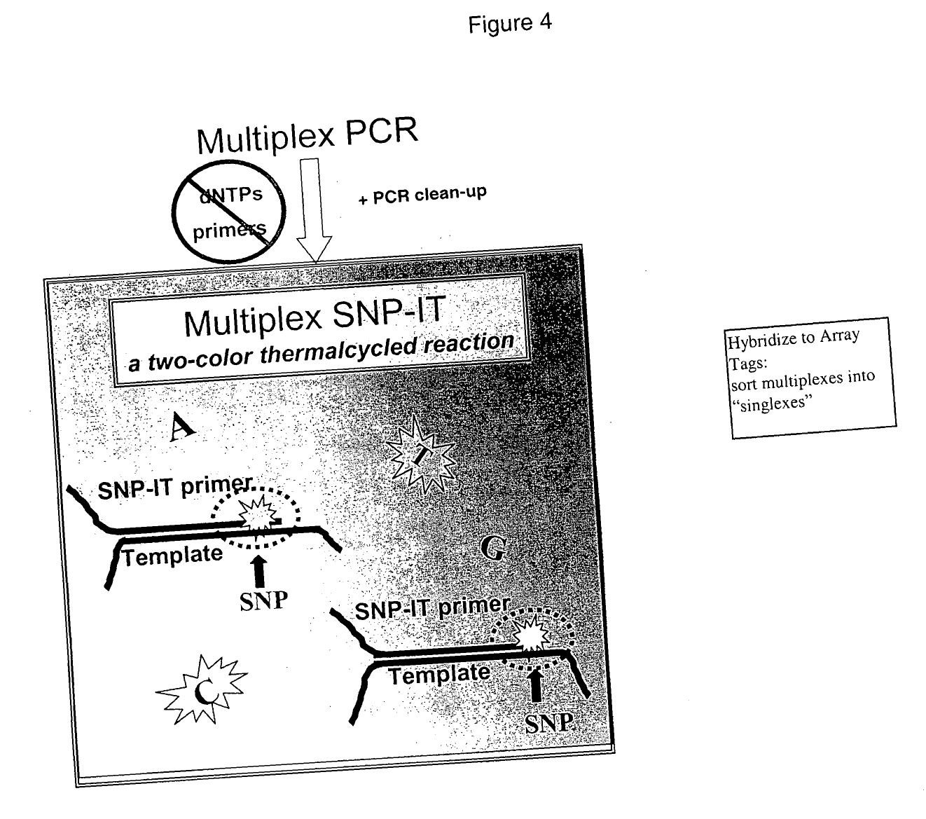

In a particularly preferred embodiment of the invention, the Affymetrix GenFlex™ tag array is used as the sorting mechanism for assessing the presence or absence of genetic alterations. A schematic diagram depicting the “tag array” methodology is provided in FIG. 4.

In another embodiment of the invention, the hybridized nucleic acids are detected by assessing one or more labels attached to the sample nucleic acids or probes. The labels may be incorporated by any of a number of means well known to those of skill in the art. However, in a preferred embodiment, the label is simultaneously incorporated during the amplification step in the preparation of the sample nucleic acids or probes. For example, polymerase chain reaction (PCR) with labeled primers or labeled nucleotides will provide a labeled amplification product. The nucleic acid (e.g., DNA) may be amplified, for example, in the presence of labeled deoxynucleotide triphosphates (dNTPs) or di-deoxynucleotide triphosphates (ddNTPs).

Alternatively, a label may be added directly to the original nucleic acid sample (e.g., mRNA, polyA mRNA, cDNA, etc.) or to the amplification product after the amplification is completed. Such labeling can result in the increased yield of amplification products and reduce the time required for the amplification reaction. Means of attaching labels to nucleic acids include, for example, nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads.TM.), fluorescent dyes (e.g., see below and, e.g., Molecular Probes, Eugene, Oreg., USA), radiolabels (e.g., ³²P, ³³P, ³⁵S, ¹²⁵I, and the like), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The detectable label can also be considered to be the unique mass of an otherwise unmodified nucleotide as well. Mass spectrometry, among other methods for determining mass differences in molecules, can distinguish primer extension reactions resulting from alternative bases having been incorporated by the polymerase, thus not necessarily requiring an exogenous label to be appended to the nucleotides.

Fluorescent moieties or labels of interest include coumarin and its derivatives, e.g., 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum dye®, fluorescent energy transfer dyes, such as thiazole orange-ethidium heterodimer, TOTAB, etc. As mentioned above, labels may also be members of a signal producing system that act in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g., antibody conjugated to a fluorescent moiety or an enzymatic moiety capable of converting a substrate to a chromogenic product, e.g., alkaline phosphatase conjugate antibody.

Alternatively, one can use different labels for each physiological source, which provides for additional assay configuration possibilities.

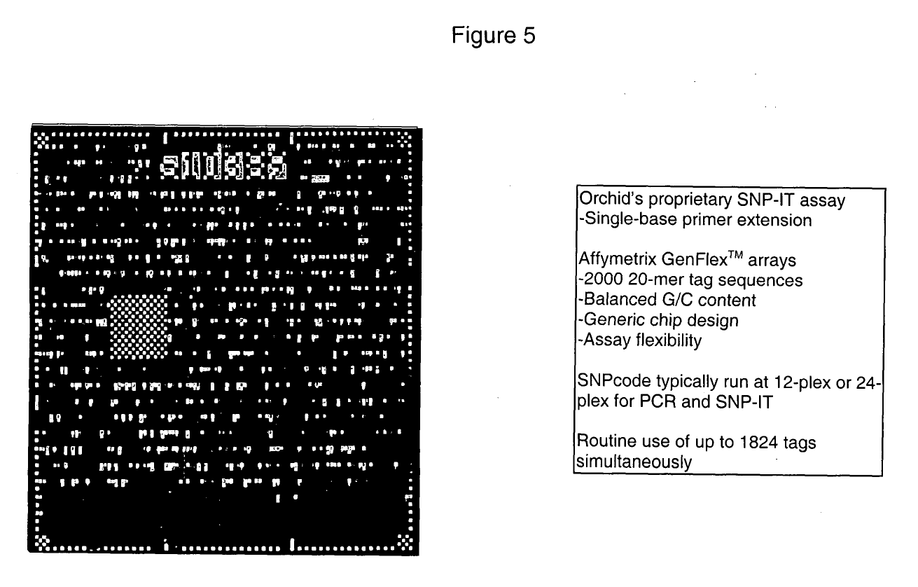

A fluorescent label is preferred because it provides a very strong signal with low background. It is also optically detectable at high resolution and sensitivity through a quick scanning procedure. FIG. 5 depicts an exemplary means for determining the presence or absence of SNPs in a patient sample based on the incorporation of flourescent tagged labels into the PCR amplification product complementary to the SNP location. The nucleic acid samples can all be labeled with a single label, e.g., a single fluorescent label. In an alternative embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another utilizing the methods of the present invention.

Suitable chromogens which may be employed include those molecules and compounds which absorb light in a distinctive range of wavelengths so that a color can be observed or, alternatively, which emit light when irradiated with radiation of a particular wave length or wave length range, e.g., fluorphores.

A wide variety of suitable dyes are available, being primarily chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

A wide variety of fluorphores may be employed either alone or, alternatively, in conjunction with quencher molecules. Fluorphores are generally preferred because by irradiating a fluorphore with light, one can obtain a plurality of emissions. Thus, a single label can provide for a plurality of measurable events.

Detectable signal can also be provided by chemiluminescent and bioluminescent sources. Chemiluminescent sources include a compound which becomes electronically excited by a chemical reaction and can then emit light which serves as the detectible signal or donates energy to a fluorescent acceptor. A diverse number of families of compounds have been found to provide chemiluminescence under a variety or conditions.

A label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

As mentioned previously, the labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. For example, labels may be attached at positions on the ribose ring or the ribose can be modified and even eliminated as desired. The base moieties of useful labeling reagents can include those that are naturally occurring or modified in a manner that does not interfere with their function. Modified bases include but are not limited to 7-deaza A and G, 7-deaza-8-aza A and G, and other heterocyclic moieties.

While the Affymetrix Genflex™ tag array is exemplified herein, other arrays for tag sequences include microparticles which have distinguishable properties, such as a variety of colors or color intensities, falling into a category of read-out often referred to as “virtual arrays”, “random arrays”, or non-spatial arrays. Flow cytometers are often used to read-out microparticles. Also, imaging by physical arrangement of the microparticles can be used, sometimes requiring the identification of each microparticle in physical space prior to utilization of the array. In an exemplary embodiment, SNPcode software by Orchid Biosciences is utilized in the method of the invention.

FIG. 6 provides a view of the SNPcode software. The so-called P-value is used as the graphical and numeric representation of the genotyping results where P is calculated as X/(X+Y), with X and Y representing the intensity of the signals matching the X and Y alleles (in this case coming from each of two fluorescence intensities with X and Y having different colors). For SNPcode, the colors are streptavidin-conjugated phycoerytrhin reacted to biotin on one nucleotide, and direct read-out of a fluorescein nucleotide for the other color. The P value represents the fraction of X allele in the calculated genotyping output, such that a P value of 1 indicates an XX homozygote, while P near zero indicates a YY homozygote, and something near 0.5 indicates a heterozygote. The screen view includes both the graphical representation of the data, in a so-called cluster graph, and also includes the data in tabular form. Individual thresholds can be set by the user, allowing simple user-driven definition of the positions of the two homozygote and one heterozygote clusters. The Y axis represents the sum of the intensity from both alleles, and can be used as equivalent to a confidence score, such that weak signals can be “failed” in comparison to stronger signals. The horizontal threshold is also user-adjustable.

Further details regarding the practice of this invention are set forth in the following examples, which are provided for illustrative purposes only and are not intended to limit the invention in any way.

### EXAMPLE I

### Typing SNPs Within and Flanking the Minimal Deleted Regions Mapping to Chromosomal Regions 1p36, 11q23 and 14q32 in Pediatric Patients with Neuroblastoma

A single nucleotide extension (SNE) assay has been developed which was used to assess the presence or absence of genetic alterations (e.g., allelic deletions) in various chromosomal regions from patients with neuroblastoma as described herein below:

I. Materials and Methods:

The following materials and methods are provided to facilitate the practice of the present invention:

A. Sample acquisition, preparation and Loss of Heterozygosity: All tissue specimens were routed through the Children's Hospital of Philadelphia (CHOP) after histopathology, DNA index and MYCN amplification status were determined. Nucleic acid extraction was performed as follows: First, 100 mg of histopathologically confirmed tumor tissue was divided and five touch preparation slides (8 imprints per slide) were made and stored at −20° C. DNA was extracted from 50 mg of tumor using anion exchange chromatography (Qiagen, Valencia Calif.). Approximately 1.75 μg of DNA/mg tumor tissue was obtained. DNA was also isolated from bone marrow or blood mononuclear cell pellets obtained from patients by anion exchange chromatography. Nucleic acids were stored at −80° C. as matched sets in 5-10 μg aliquots depending on the total yield. Quality assurance was maintained by spot checks for efficiency of restriction enzyme digestion, PCR, and RT-PCR amplification.

Loss of heterozygosity (LOH) was assessed by standard microsatellite analysis using gel-based methods as well as by a solution-phase reporter/quencher assay.

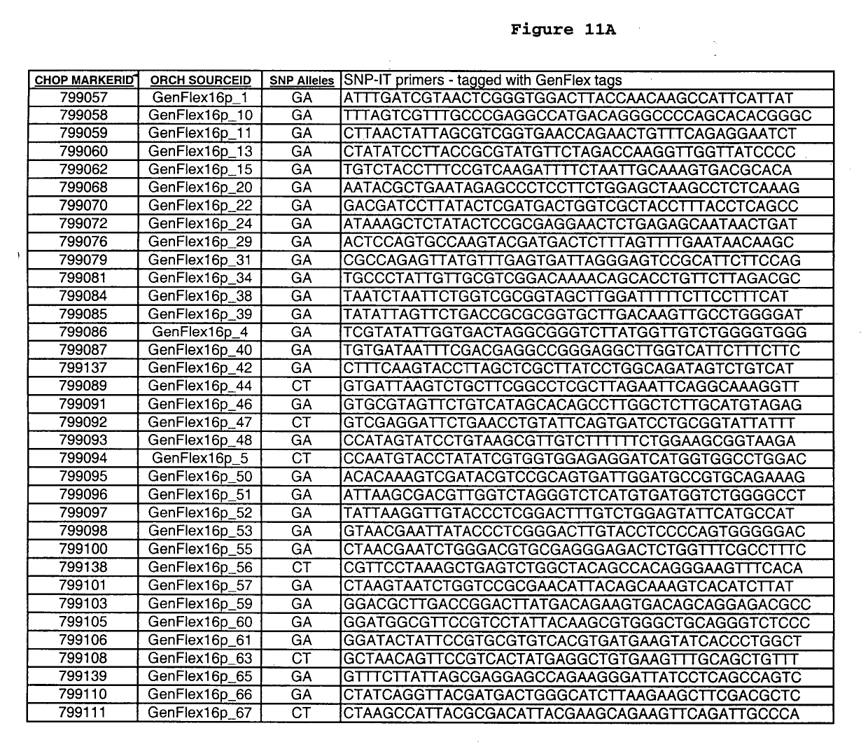

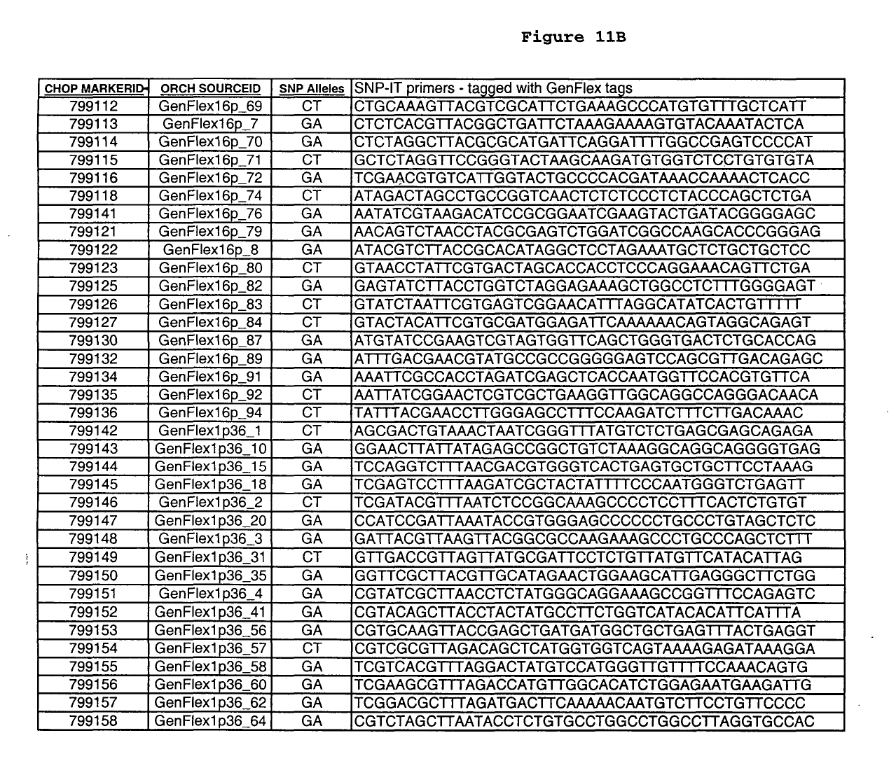

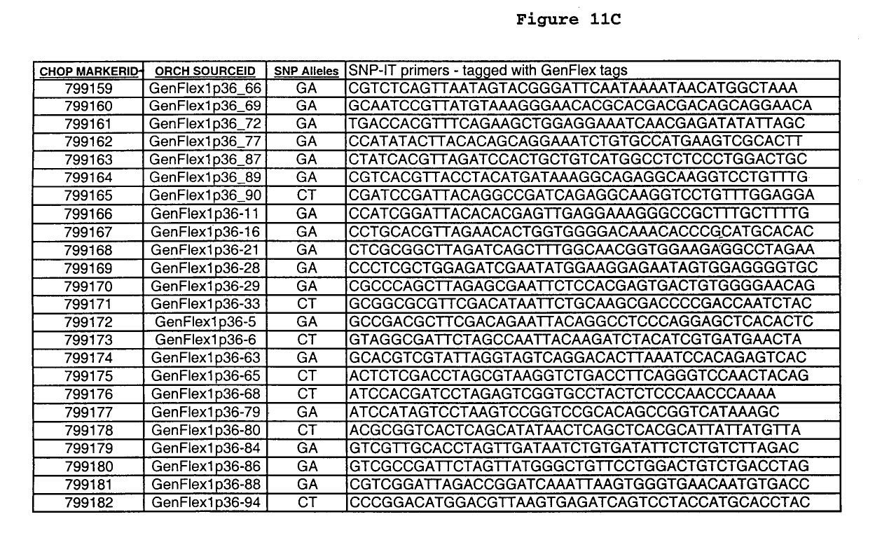

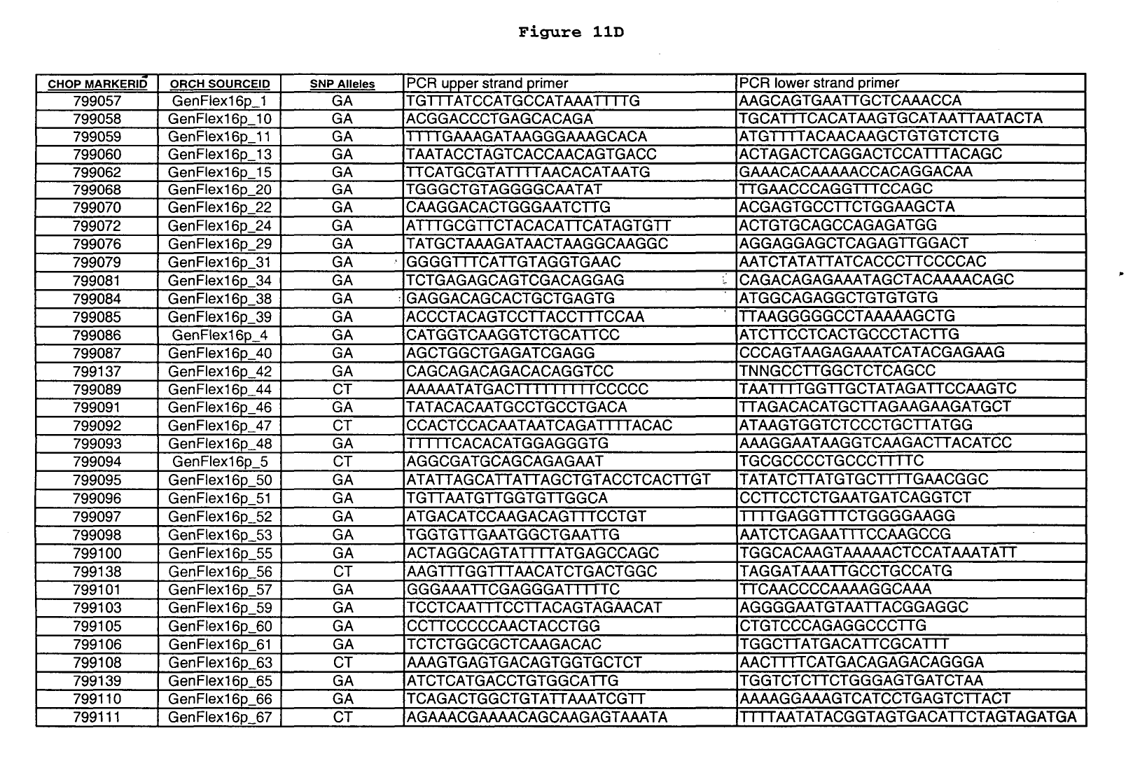

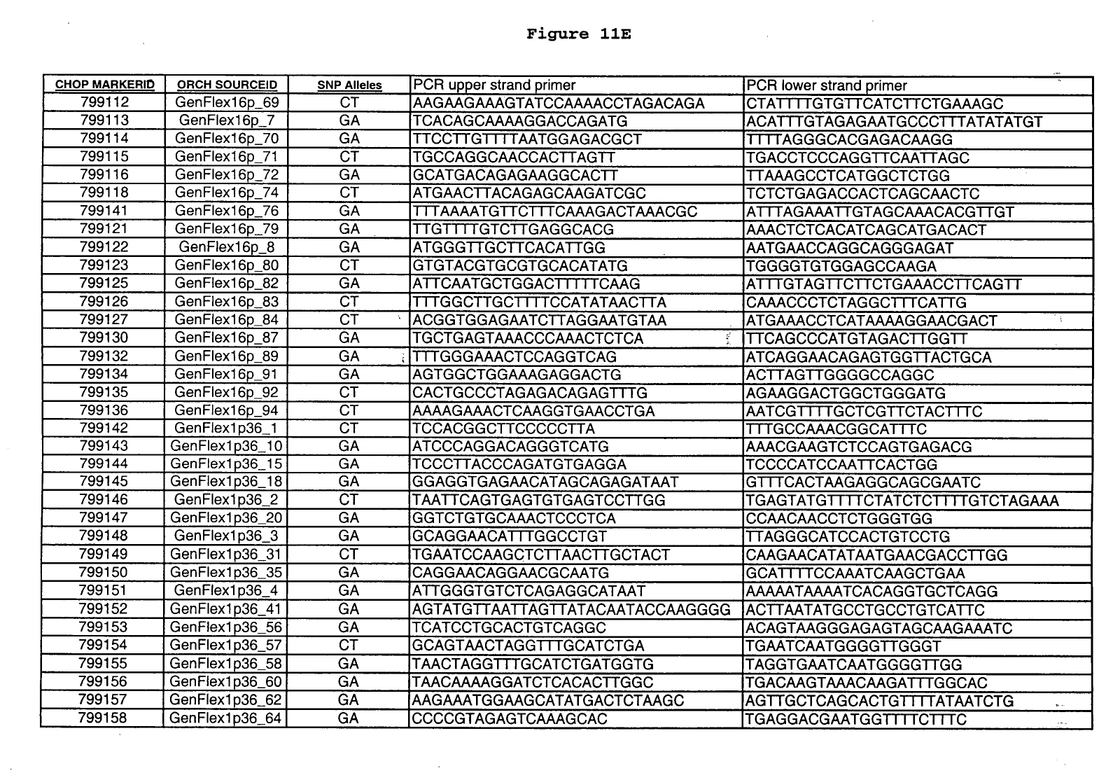

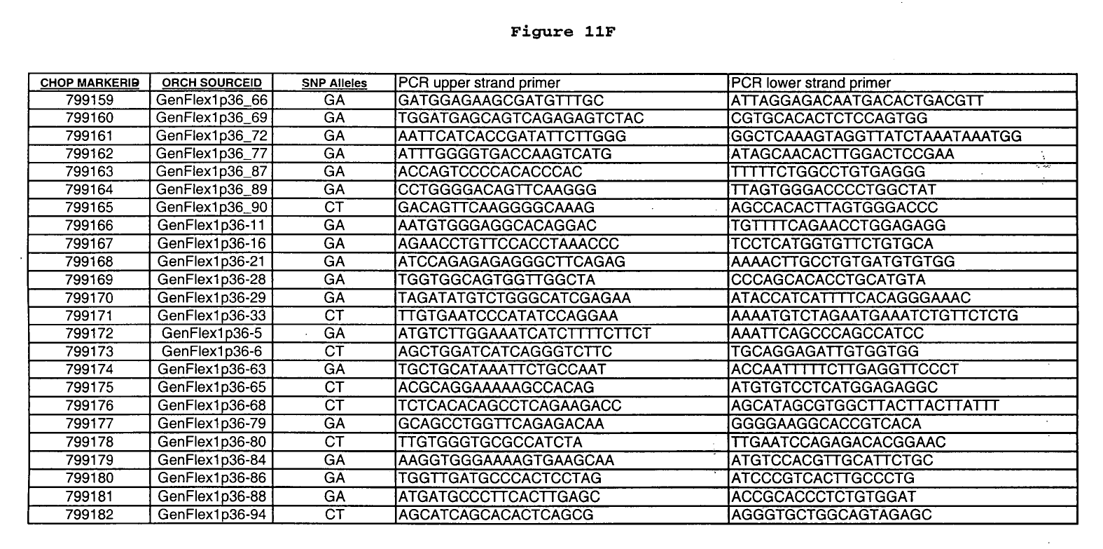

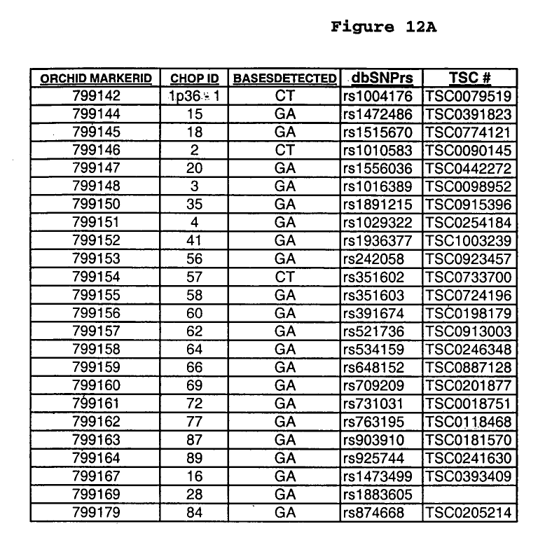

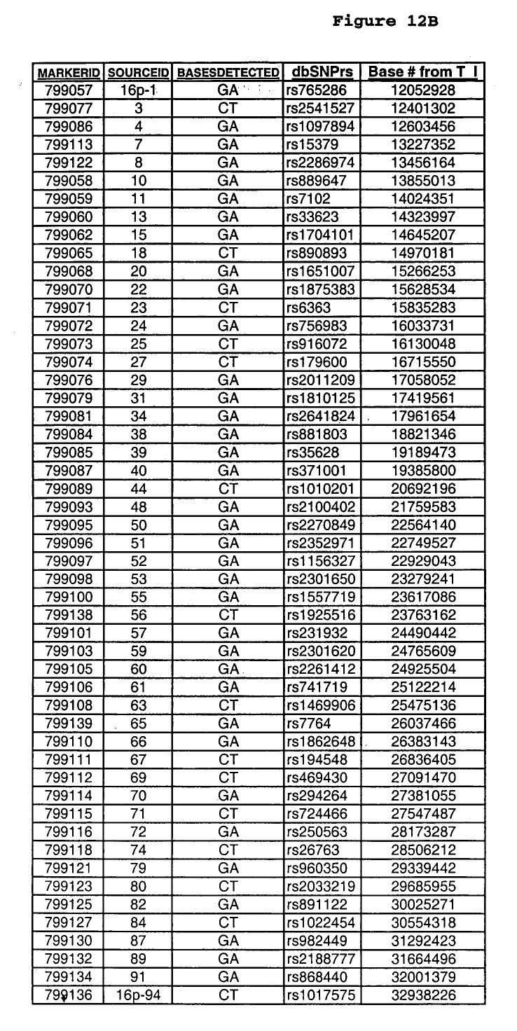

B. SNP mapping: Single nucleotide polymorphisms (SNPs) within and flanking the 1p36 chromosomal minimal deleted region associated with neuroblastoma were identified from the Orchid Bioscienses SNP databases. The PCR and SNE reactions described above were performed in mutiplex reactions on this set of SNPs. Exemplary oligonucleotide primers utilized are provided in FIGS. 11A-11F (SEQ ID NOS: 1-282). PCR products encompassing these SNPs, derived from an input of total genomic DNA, were used as targets for interrogation. In addition, arrays were constructed containing multiple patient samples in order to screen for LOH of an informative SNP mapping within the minimal deleted regions. Normal and tumor tissue from the same patient were analyzed by array-based SNE. FIGS. 12A and 12B provide the SNPs examined from the 1p and 16p regions of chromosomes 1 and 16, respectively.

C. PCR Reactions: Genomic DNA was used to amplify a region encompassing the SNPs of interest through PCR. Typically the PCR reaction was multiplexed where 12 SNP sequences were amplified at the same time in the same well. These SNPs, and their primers, were grouped together by extension mix, i.e. SNPs of the same two alleles. The PCR reaction was set up as follows:

Final Concentration

| | |
|---|---|
| PCR Upper Primer | 50 nM |
| Lower PCR Primer | 50 nM |
| DNTPs | 75 μM each |
| KCl | 50 mM |
| Tris-HCl, pH 8.3 | 10 mM |
| MgCl₂ | 5 mM |
| Taq Gold ® | 2.5 U/25 μl |
| Genomic DNA | 10 ng/25 μl |

PCR amplification conditions were as follows:

| | |
|---|---|
| Step 1. | 95° C. for 5:00 |
| Step 2. | 95° C. for 0:30 |
| Step 3. | 50° C. for 0:55 |
| Step 4. | 72° C. for 0:30 |
| Step 5. | Go to step 2, 4 times |
| Step 6. | 95° C. for 0:30 |
| Step 7. | 50° C. for 0:55 + 0.2° C./cycle |
| Step 8. | 72° C. for 0:30 |
| Step 9. | Go to step 6, 24 times |
| Step 10. | 95° C. for 0:30 |
| Step 11. | 55° C. for 0:55 |
| Step 12. | 72° C. for 0:30 |
| Step 13. | Go to 10, 4 times |
| Step 14. | 72° C. for 7:00 |
| Step 15. | 4° C. hold forever |

Following PCR amplification, the product was cleaned with Exonuclease I and Shrimp Alkaline Phosphatase (SAP). Exonuclease I digested away the excess PCR primers and SAP removed the free nucleotides from the PCR reaction. The digestion was set up in the thermocycler at 37° C. for 30 minutes and the enzymes were then heat-inactivated at 95° C. for 10 minutes.

Single-well, two-color SNP-IT single-base extensions were set up using two labeled nucleotide terminators, bearing either Fluorescein or Biotin, with labels on the bases matching the alleles of the SNPs being tested. In this set, all SNPs were selected to have either T/C or A/G alternative alleles. The SNP-IT reaction typically consisted of the following reaction mixture:

| Reagent | Volume (μl) |
|---|---|
| Cleaned PCR Product | 12 |
| Tag-SNP IT ™ Primer Pool | 2.5 |
| 1M Tris HCl, pH 9.5 | 1.7 |
| 100 mM MgCl2 | 2.2 |
| Fluorescein, 52 μm | 0.33 |
| Biotin, 15.6 μm | 0.33 |
| Unlabeled nucleotide, 20.8 μm | 0.33 |
| Unlabeled nucleotide, 20.8 μm | 0.33 |
| Thermosequenase ® (32 U/μl) | 0.078 |
| Water | 10.702 |
| Total Volume | 33 |

The extension reactions were carried out using the following method:

| | |
|---|---|
| Step 1. | 96° C. for 3:00 min |
| Step 2. | 94° C. for 0:20 sec |
| Step 3. | 40° C. for 0:11 sec |
| Step 4. | Go to Step 2, 45 times |
| Step 5. | 4° C. hold forever |

The reactions were pooled together into a single tube for precipitation, as follows: 1.2 ml pre-chilled (−20° C.) absolute ethanol was added to a 1.5 ml eppendorf tube and placed on ice. 19.85 μl of 8 M LiCl was then added along with 33 μl of glycogen (100 μg/ml). The SNPcode reactions were then pooled into the ethanol solution and vortexed. The reaction pool was centrifuged at 16,000×g for 15 minutes at room temperature and then the supernatant was removed. The open tubes were then placed in a 42° C. oven for approximately 10 minutes. The pellets were then resuspended in 38 μl molecular biology grade water.

D. GenFlex Microarray: The sample was prepared for hybridization and then applied to the Affymetrix GenFlex™ chip. 50 μl of 2× Hybridization Buffer was added to the 38 μl of resuspended pellet along with 10 μl of 10× Two Color Hybridization Controls. 2 μl of 50× Denhardt's Solution was then added and the solution was vortexed. The samples were spun briefly to collect the sample at the bottom of the tube. The samples were heated to 100° C. for 10 minutes and snap cooled on ice for 2 minutes. The samples were then spun at max speed for 2 minutes. The samples were then applied to the chip as per manufacturer's instructions, covering each chip with 100 μl of the prepared sample. The chips were placed in a GeneChip Hybridization Oven 640 at 42° C. for 2 hours, and rotated at 50 rpm.

The chip was run through the Affymetrix fluidics station using protocol “GenFlex”, with the following wash and stain buffers: Non-Stringent Wash Buffer (A) (final 6× (1 M) SSPE, 0.01% Tween®-20); Stringent Wash Buffer (B) (final 3× (0.5 M) SSPE, 0.01% Tween®-20); and Stain buffer (final 6×SSPE, 1× Denhardt's solution, 0.01% Tween®-20, 5 μg/ml Streptavidin, 5 μg/ml SAPE [Streptavidin Phycoerythrin]).

Data were extracted using the SNPcode software by Orchid Biosciences. The basic algorithm balanced the relative contribution of the two colors in the Affymetrix imaging system using the intensities from hybridization controls. Once balanced, the P value (percent of allele X in the reaction), was calculated as X/(X+Y), with X and Y representing the corrected fluorescence intensities of each SNP allele in the reaction. The left and right clusters represented the Y homozygote and X homozygote genotypes, respectively, and the central cluster represented heterozygotes.

II. Results:

SNPs mapping to various chromosomal regions commonly involved in human neuroblastoma were simultaneously typed from normal tissue (e.g., peripheral blood) and tumor tissue derived from the same patient to assess LOH of informative SNPs. Individuals whose deleted region encompassed all markers or whose SNPs were not informative (e.g., homozygous in normal tissue) were further typed for additional SNPs mapping at increasing distances from the minimal deleted region (MDR) to rapidly genotype these individuals for deletion location and extent.

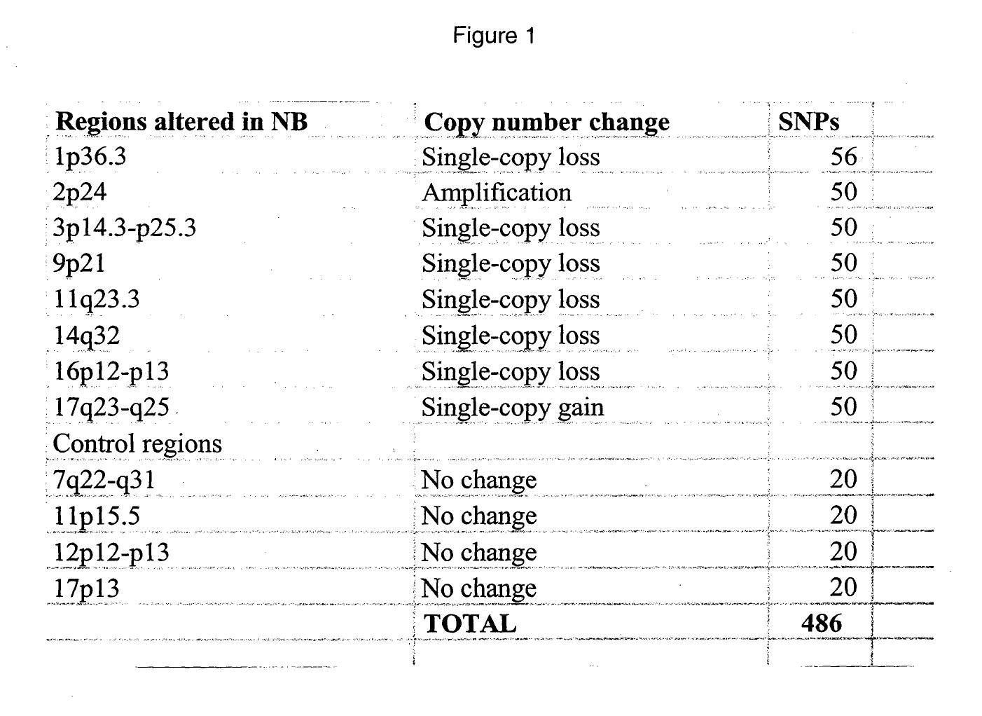

FIG. 1 provides a list of eight chromosomal regions commonly involved in human neuroblastoma tumorigenesis. Gain or loss of genetic material (copy number change) at each locus correlates with tumor aggressiveness and patient outcome. Alterations in any of these chromosomal regions is associated with an increased chance of metastatic disease. Also listed is the number of validated SNPs used in this assay at each neuroblastoma region, and 4 control regions, to detect copy number aberrations. A complete list of the SNPs examined from the 1p and 16p regions of chromosomes 1 and 16, respectively are provided in FIGS. 12A and 12B.

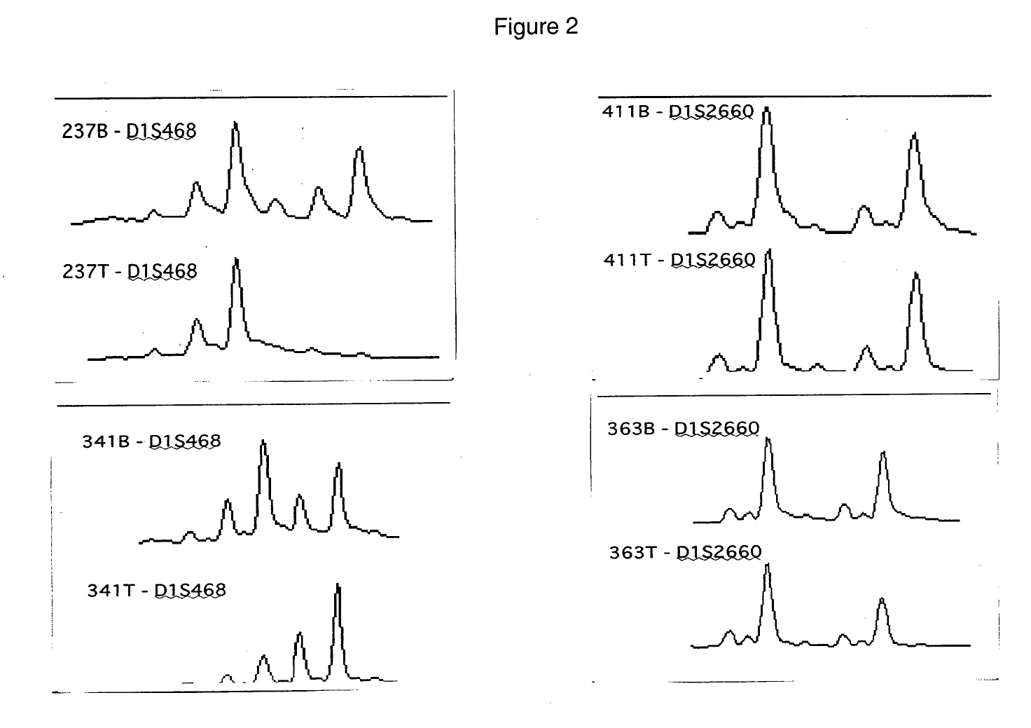

LOH analyses using conventional PCR-based genotyping at simple tandem repeat polymorphic (STRP) loci located on the distal short arm of chromosome 1 (1p36) were performed and the results are illustrated in FIG. 2. The four panels show data from four separate tumor (T) and blood (B) paired DNA samples at two different STRP loci (D1S468 and D1S2660). At D1S468, sample 273 (top left panel) shows heterozygosity (two peaks) in the blood sample, one of which is lost in the tumor sample, demonstrating LOH at this locus. Sample 341 also shows LOH at this locus, but samples 411 and 363 show no evidence for LOH. These data were generated in uniplex PCR reactions sequentially and only 3 loci were examined per patient in a time consuming process.

Neuroblastoma Tag Array:

A strategy was devised to build the first generation neuroblastoma-specific tag array. Previously, a critical region at 1p36.3 was mapped that is deleted in all neuroblastoma specimens that show LOH. Fifty SNPs flanking this region were identified in the Orchid database. As a negative control, markers at 16 p12-13 were used. This tag array was tested for sensitivity and specificity for detection of 1p deletions using human neuroblastoma cell lines as primary tumor specimens, each with a matched constitutional DNA sample as a control.

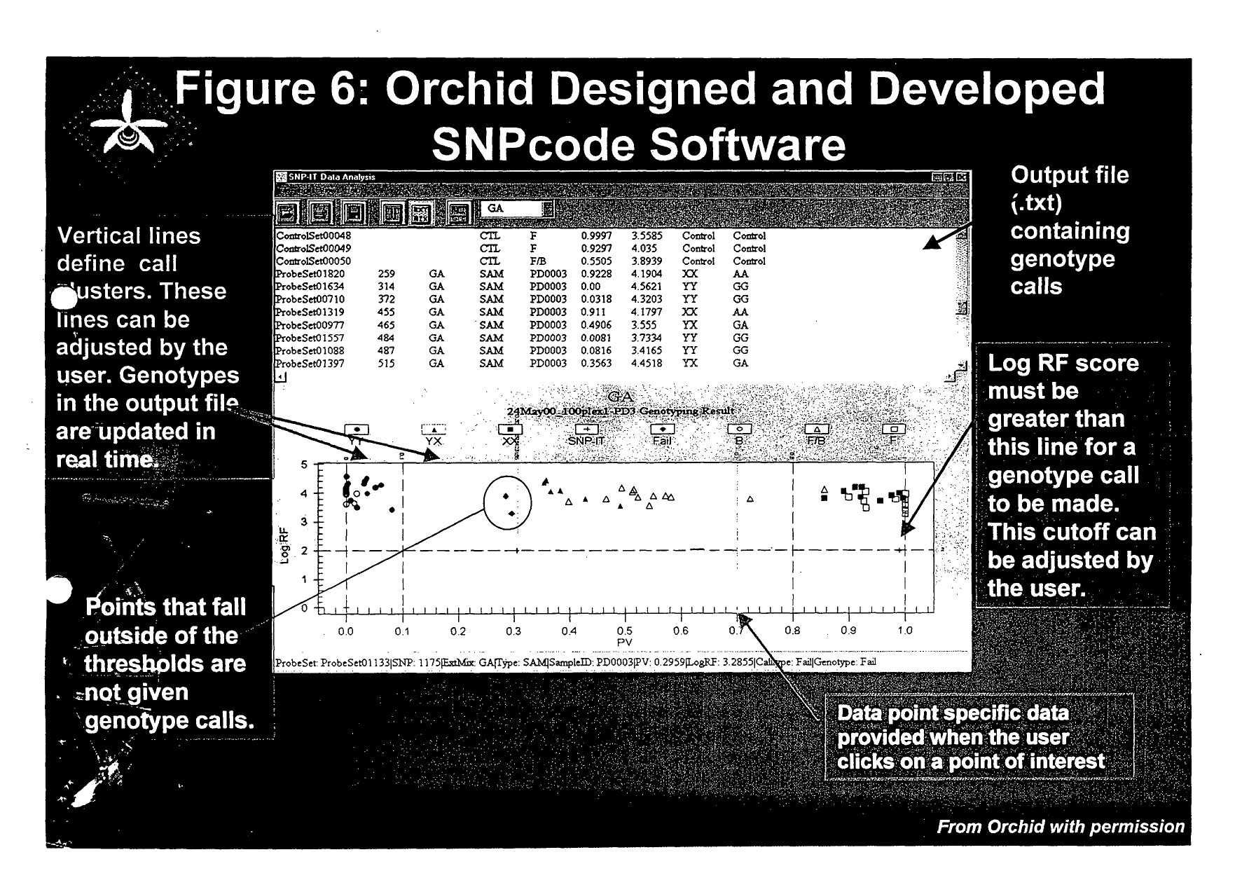

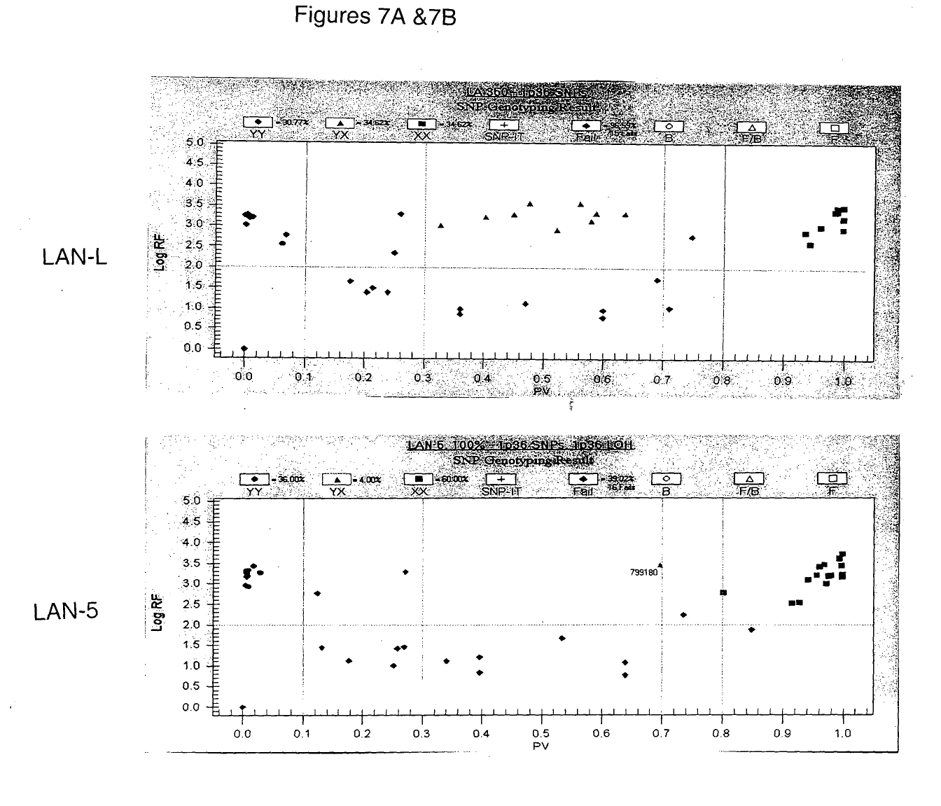

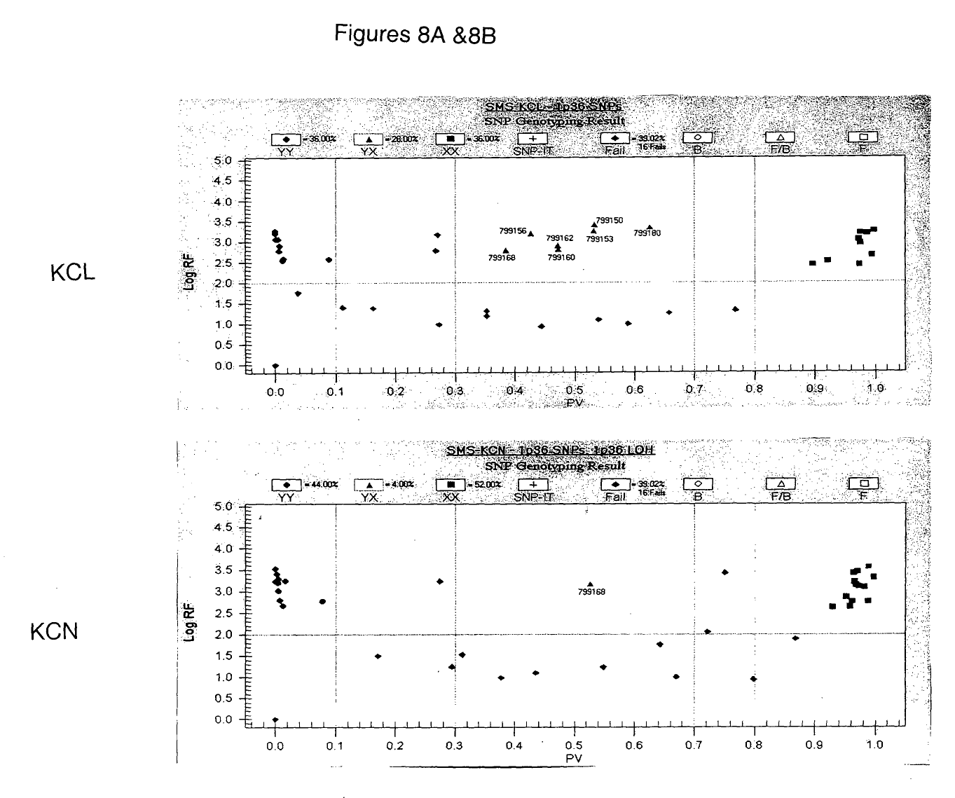

FIGS. 7A and 7B show the results obtained using the method of the invention on DNA isolated from the neuroblastoma cell line, LAN-5, and a matched control, LAN-L (lymphocyte-derived DNA from the patient from whom the LAN-5 neuroblastoma cell line was established). Each fluorescence signal is shown as a colored symbol, with intensity of signal depicted on a log scale. Y-axis shows relative fluorescence (RF) intensity and X-axis the “percentage value” (PV) of fluorescence at 530 and 570 nm wavelengths. SNPs with RF<2.0 were considered failed. In the blood-derived LAN-L sample, there was the expected number of SNPs showing constitutional homozygosity (PVs near 0 or 1, with 9 SNPs showing constitutional heterozygosity (i.e. informative SNPs). The tumor-derived LAN-5 sample showed conversion of each of these SNPs to “homozygosity” due to LOH by hemizygous deletion of one allele. SNP 799180 was subsequently shown to have repeats elsewhere in the genome and was replaced in later versions of the chip. These data document the ability to detect LOH using this tag array system. FIGS. 8A and 8B show similar results obtained using the method of the invention on DNA isolated from the neuroblastoma cell line, KCN, and its matched control, KCL.

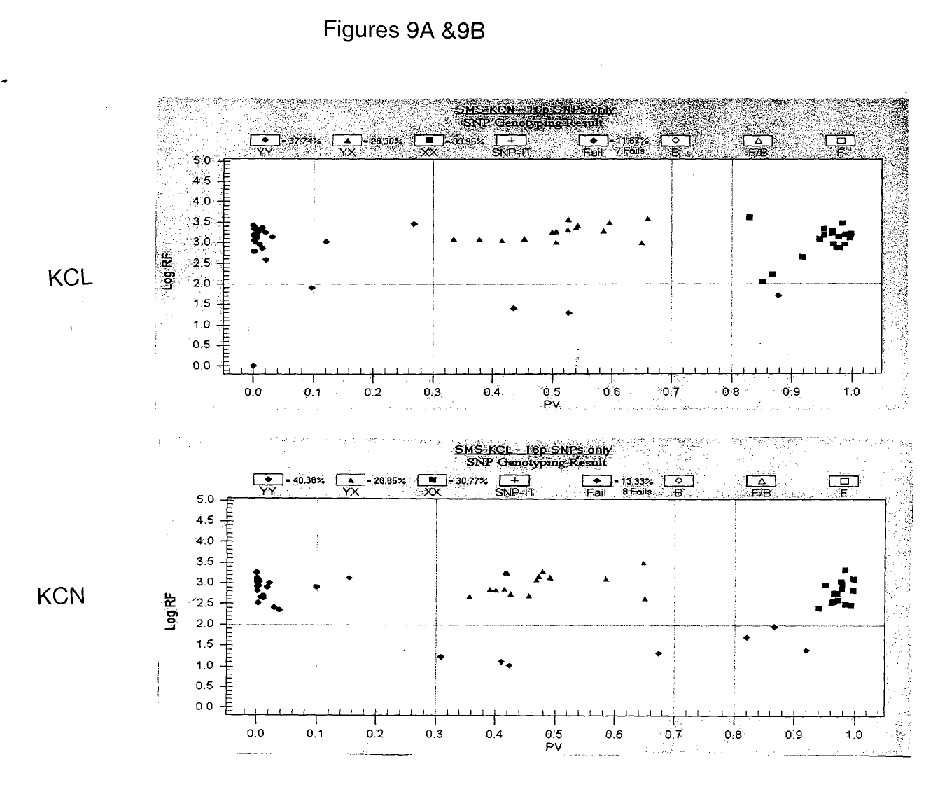

FIGS. 9A and 9B show a negative control experiment. The KCN neuroblastoma cell line was assessed for deletions at the 16 p12-13 control region using the method of the invention. Previous work demonstrated that the 16 p12-13 locus was present in two copies (i.e., normal) in the KCN cell line. As expected, the results demonstrated no LOH in the tumor-derived sample when compared to the blood-derived KCL sample at multiple SNP markers mapping to 16 p12-13.

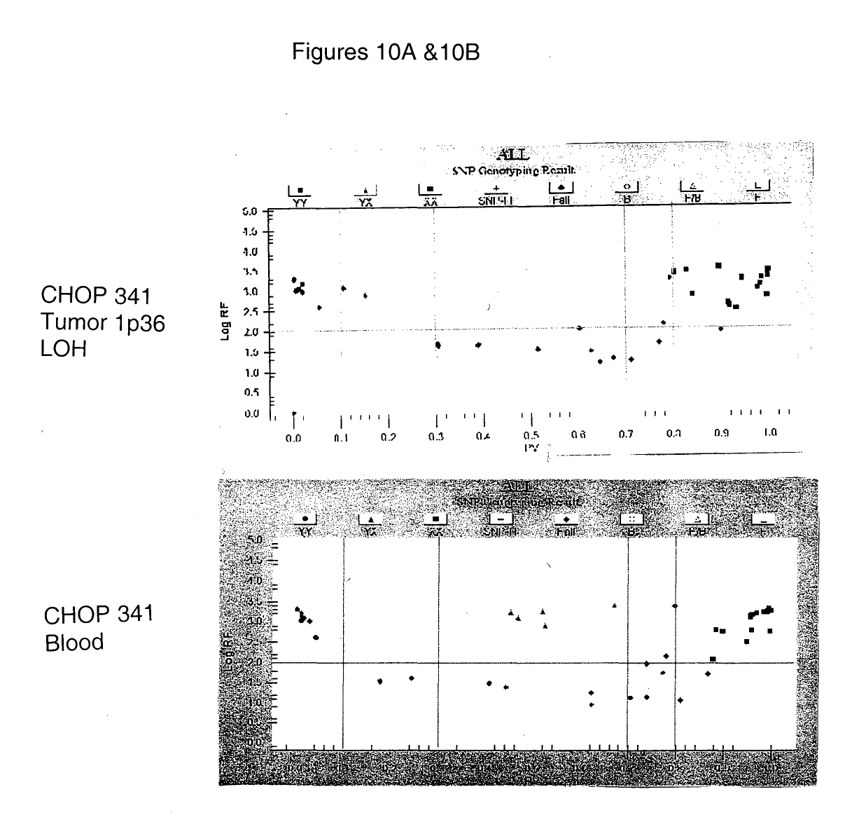

FIGS. 10A and 10B show the LOH results obtained using tumor (FIG. 10A) and blood-derived (FIG. 10B) genomic DNA samples from one neuroblastoma patient (CHOP 341). The Y-axis shows relative fluorescence (RF) intensity and the X-axis the “percentage value” (PV) of fluorescence at 530 and 570 nm wavelengths. SNPs with RF<2.0 were considered failed. In the blood sample, there was the expected number of SNPs showing constitutional homozygosity (PVs near 0 or 1, with 5 SNPs showing constitutional heterozygosity (i.e. informative SNPs). The tumor sample showed conversion of each of these SNPs to “homozygosity” due to LOH by hemizygous deletion of one allele. More recent versions of this chip utilized SNPs with much higher heterozygosity scores increasing the number of informative SNPs available for LOH analysis. These results demonstrate the ability to detect LOH in real patient-derived tumor samples. Dilution experiments have also been performed which demonstrated that LOH can be detected in a primary tumor specimen “contaminated” with up to 30-40% normal tissue (data not shown).

### EXAMPLE II

Parallel Region-Specific Evaluation of Multiple Genomic Copy Number Alterations in Human Neuroblastoma Specimens

The following experiments are provided to adapt the array-based SNE assay for parallel region-specific evaluation of multiple genomic copy number alterations in human neuroblastoma specimens. This array-based SNE assay will allow for the parallel allelotyping of four separate patient-derived tumor DNA specimens simultaneously. Redundancy of SNPs, which is a major limitation for current approaches to detecting LOH, will obviate the need for a matched control sample. Each specimen will be assayed for copy number alterations at nine separate genomic regions that reproducibly show allelic loss or gain in human neuroblastoma, as well as four control regions (See Table 1).

TABLE 1
Genomic regions to be assayed in a neuroblastoma-specific array-based
SNE system

| Regions with | | |
|---|---|---|
| Copy-Number | Type of Copy | Number SNPs |
| Changes¹ | Number Change² | To Be Assayed |
| 1p36.3 | Single-copy loss | 45 |
| 2p24 | Amplification | 45 |
| 3p14.3-p25.3 | Single-copy loss | 45 |
| 4p15-p16 | Single-copy loss | 45 |
| 9p21 | Single-copy loss | 45 |
| 11q23.3 | Single-copy loss | 45 |
| 14q32 | Single-copy loss | 45 |
| 16p12-p13 | Single-copy loss | 45 |
| 17q23-q25 | Single-copy gain | 45 |
| Control regions³ | | |
| 7q22-q31 | No change | 20 |
| 11p15.5 | No change | 20 |
| 12p12-p13 | No change | 20 |
| 17p13 | No change | 20 |

| | |
|---|---|
| TOTAL | 485 |

¹Genomic regions showing non-random allelic gain or loss in human neuroblastomas;
²Type of allelic alteration observed;
³Regions that are infrequently altered in human neuroblastoma (<5% cases) and/or copy number status is relevant to a region listed above (e.g., unbalanced gain of 17q material in relation to 17p material).

45 tightly linked SNPs have been identified within each of the nine genomic regions of interest and 20 SNPs from each of the four “control” regions. The SNPs were selected by sequence-derived chromosomal location and by choosing markers with heterozygosity scores greater than 30%.

For each SNP, four separate SNE-Tag chimeric oligonucleotides will be designed. This will allow for the parallel analysis of four separate specimens on each microarray chip (485 SNPs per sample, 1940 unique Tags/chip). This enhanced throughput will greatly reduce cost thus making this approach more likely to be clinically applicable in the future. Hybridization data will be parsed in a region-specific manner and the final output of allelic gain or loss will be made based upon the signal intensities at each scanned wavelength averaged for all SNP makers within a region.

For these experiments, genomic tumor DNA will be amplified in a 12-plex multiplex PCR reaction at region-specific SNPs that have been previously defined. PCR products will then be extended in a 72-plex solution-phase reaction using 2 dye-tagged ddNTP and 2 standard non fluorescently labeled ddNTP. This reaction will be performed with a chimeric oligonucleotide with 5′ complimentarity to a specific tag on the chip and 3′ complimentarity to the SNP amplimer, with the 3′ nucleotide ending one base before the polymorphic site. The extended products will then be interrogated in a solid-phase reaction using a GenFlex™ chip. The GenFlex™ Tag Array will enable the interrogation of up to 2000 nucleic acid reaction products and 50 control oligonucleotides.

After hybridization of pooled SNE products to the tag array, fluorescent signals will quantitatively represent allelic representation for each SNP. The analyzed fluorescent intensity values will then be imported into SNPCode (Orchid BioScience, Princeton, N.J.) for signal deconvolution and genotyping using a proprietary algorithm. Plots will be generated with log of relative fluorescent intensity (Log RF) and the percentage ratio of the two fluors (PV) on y- and x-axes, respectively. By default, any value<2.0 on the Y-axis will be scored as “Failed” whereas on the X-axis, values between 0.1 to 0.3 and 0.7 to 0.8 will also be scored as “Failed”. By default again, on the X-axis, any values in between 0.0 to 0.1 will be scored as “XX” and values between 0.3 to 0.7 will be scored as “YX”, and values between 0.8 to 1.0 will be scored as “YY”.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for determining the presence or absence of at least one genetic alteration in a target nucleic acid for the diagnosis and management of malignant disease, comprising:
a) providing a target nucleic acid from a patient sample, said target nucleic acid having a predetermined sequence in the normal population;
b) assessing said target nucleic acid for the extent of loss of heterozygosity relative to predetermined loci, increased loss of heterozygosity, being correlated with enhanced tumor invasiveness and metastasis.

2. The method as claimed in claim 1, wherein said genetic alteration is selected from the group consisting of inversion, deletion, duplication, and insertion of at least one nucleotide in said sequence.

3. The method of claim 1, wherein said target nucleic acid is assessed for genetic alterations via a method selected from the group consisting of restriction enzyme mapping, hybridization of allele specific probes, oligomer ligation, DNA sequencing and quantitative PCR.

4. The method as claimed in claim 1, wherein said malignancy is neuroblastoma and said genetic alteration is a single copy loss of a single nucleotide polymorphism at the 1p36.3 region of chromosome 1, said loss being associated with increased metastasis and poor prognosis.

5. The method as claimed in claim 4, wherein said Loss of heterozygosity occurs on chromosome 1 and said single nucleotide polymorphism comprises at least one of the single nucleotide polymorphisms set forth in FIG. 12.

6. A method for determining the presence or absence of at least one specific nucleotide in a target nucleic acid for the diagnosis and management of malignant disease, the method comprising the steps of:
(a) providing a detectable amount of a target nucleic acid polymer isolated from a chromosomal region known to be associated with malignancy in a single stranded form,
(b) hybridizing the detectable amount of the nucleic acid polymer with one or more oligonucleotide primers, wherein each primer has a nucleotide sequence that is complementary to a sequence in the target nucleic acid polymer, such that when the primer is hybridized to the target nucleic acid polymer, the 3′ end of the primer binds to a nucleotide flanking the specific nucleotide at the defined site in the target nucleic acid,
(c) exposing the hybridized nucleic acid polymer to a polymerization agent in a mixture containing at least one deoxynucleotide, said deoxynucleotide comprising a detectable label, and one or more chain terminating nucleotide analogues, such that a detectable primer extension product is formed if the labeled deoxynucleotide is complementary to the specific nucleotide at the defined site;
(d) analyzing the polymerization mixture of step (c) for the presence or absence of the primer extension product containing the labeled deoxynucleotide at the 3′ end thereof, whereby the identity of the specific nucleotide at the defined site is determined; and
(e) assessing said target nucleic acid for loss of heterozygosity at said at least one single nucleotide loci, the degree of loss of heterozygosity being correlatable with increased tumor invasiveness and poor patient prognosis.

7. The method of claim 6, wherein said target nucleic acid of step e) is compared to the chromosomal region of step b) which lacks genetic alterations associated with cancer.

8. The method as claimed in claim 6, wherein said malignancy is neuroblastoma and said genetic alteration is a single copy loss of a single nucleotide polymorphism at the 1p36.3 region of chromosome 1, said loss being associated with increased metastasis and poor prognosis.


SEQUENCE LISTING (282 sequences)

SEQ ID NO: 1 - DNA, 45 bp, Artificial Sequence
  Primer
atttgatcgtaactcgggtggacttaccaacaagccattcattat

SEQ ID NO: 2 - DNA, 45 bp, Artificial Sequence
  Primer
tttagtcgtttgcccgaggccatgacagggccccagcacacgggc

SEQ ID NO: 3 - DNA, 45 bp, Artificial Sequence
  Primer
cttaactattagcgtcggtgaaccagaactgtttcagaggaatct

SEQ ID NO: 4 - DNA, 45 bp, Artificial Sequence
  Primer
ctatatccttaccgcgtatgttctagaccaaggttggttatcccc

SEQ ID NO: 5 - DNA, 45 bp, Artificial Sequence
  Primer
tgtctacctttccgtcaagattttctaattgcaaagtgacgcaca

SEQ ID NO: 6 - DNA, 45 bp, Artificial Sequence
  Primer
aatacgctgaatagagccctccttctggagctaagcctctcaaag

SEQ ID NO: 7 - DNA, 45 bp, Artificial Sequence
  Primer
gacgatccttatactcgatgactggtcgctacctttacctcagcc

SEQ ID NO: 8 - DNA, 45 bp, Artificial Sequence
  Primer
ataaagctctatactccgcgaggaactctgagagcaataactgat

SEQ ID NO: 9 - DNA, 45 bp, Artificial Sequence
  Primer
actccagtgccaagtacgatgactctttagttttgaataacaagc

SEQ ID NO: 10 - DNA, 45 bp, Artificial Sequence
  Primer
cgccagagttatgtttgagtgattagggagtccgcattcttccag

SEQ ID NO: 11 - DNA, 45 bp, Artificial Sequence
  Primer
tgccctattgttgcgtcggacaaaacagcacctgttcttagacgc

SEQ ID NO: 12 - DNA, 45 bp, Artificial Sequence
  Primer
taatctaattctggtcgcggtagcttggatttttcttcctttcat

SEQ ID NO: 13 - DNA, 45 bp, Artificial Sequence
  Primer
tatattagttctgaccgcgcggtgcttgacaagttgcctggggat

SEQ ID NO: 14 - DNA, 45 bp, Artificial Sequence
  Primer
tcgtatattggtgactaggcgggtcttatggttgtctggggtggg

SEQ ID NO: 15 - DNA, 45 bp, Artificial Sequence
  Primer
tgtgataatttcgacgaggccgggaggcttggtcattctttcttc

SEQ ID NO: 16 - DNA, 45 bp, Artificial Sequence
  Primer
ctttcaagtaccttagctcgcttatcctggcagatagtctgtcat

SEQ ID NO: 17 - DNA, 45 bp, Artificial Sequence
  Primer
gtgattaagtctgcttcggcctcgcttagaattcaggcaaaggtt

SEQ ID NO: 18 - DNA, 45 bp, Artificial Sequence
  Primer
gtgcgtagttctgtcatagcacagccttggctcttgcatgtagag

SEQ ID NO: 19 - DNA, 45 bp, Artificial Sequence
  Primer
gtcgaggattctgaacctgtattcagtgatcctgcggtattattt

SEQ ID NO: 20 - DNA, 45 bp, Artificial Sequence
  Primer
ccatagtatcctgtaagcgttgtcttttttctggaagcggtaaga

SEQ ID NO: 21 - DNA, 45 bp, Artificial Sequence
  Primer
ccaatgtacctatatcgtggtggagaggatcatggtggcctggac

SEQ ID NO: 22 - DNA, 45 bp, Artificial Sequence
  Primer
acacaaagtcgatacgtccgcagtgattggatgccgtgcagaaag

SEQ ID NO: 23 - DNA, 45 bp, Artificial Sequence
  Primer
attaagcgacgttggtctagggtctcatgtgatggtctggggcct

SEQ ID NO: 24 - DNA, 45 bp, Artificial Sequence
  Primer
tattaaggttgtaccctcggactttgtctggagtattcatgccat

SEQ ID NO: 25 - DNA, 45 bp, Artificial Sequence
  Primer
gtaacgaattataccctcgggacttgtacctccccagtgggggac

SEQ ID NO: 26 - DNA, 45 bp, Artificial Sequence
  Primer
ctaacgaatctgggacgtgcgagggagactctggtttcgcctttc

SEQ ID NO: 27 - DNA, 45 bp, Artificial Sequence
  Primer
cgttcctaaagctgagtctggctacagccacagggaagtttcaca

SEQ ID NO: 28 - DNA, 45 bp, Artificial Sequence
  Primer
ctaagtaatctggtccgcgaacattacagcaaagtcacatcttat

SEQ ID NO: 29 - DNA, 45 bp, Artificial Sequence
  Primer
ggacgcttgaccggacttatgacagaagtgacagcaggagacgcc

SEQ ID NO: 30 - DNA, 45 bp, Artificial Sequence
  Primer
ggatggcgttccgtcctattacaagcgtgggctgcagggtctccc

SEQ ID NO: 31 - DNA, 45 bp, Artificial Sequence
  Primer
ggatactattccgtgcgtgtcacgtgatgaagtatcaccctggct

SEQ ID NO: 32 - DNA, 45 bp, Artificial Sequence
  Primer
gctaacagttccgtcactatgaggctgtgaagtttgcagctgttt

SEQ ID NO: 33 - DNA, 45 bp, Artificial Sequence
  Primer
gtttcttattagcgaggagccagaagggattatcctcagccagtc

SEQ ID NO: 34 - DNA, 45 bp, Artificial Sequence
  Primer
ctatcaggttacgatgactgggcatcttaagaagcttcgacgctc

SEQ ID NO: 35 - DNA, 45 bp, Artificial Sequence
  Primer
ctaagccattacgcgacattacgaagcagaagttcagattgccca

SEQ ID NO: 36 - DNA, 45 bp, Artificial Sequence
  Primer
ctgcaaagttacgtcgcattctgaaagcccatgtgtttgctcatt

SEQ ID NO: 37 - DNA, 45 bp, Artificial Sequence
  Primer
ctctcacgttacggctgattctaaagaaaagtgtacaaatactca

SEQ ID NO: 38 - DNA, 45 bp, Artificial Sequence
  Primer
ctctaggcttacgcgcatgattcaggattttggccgagtccccat

SEQ ID NO: 39 - DNA, 45 bp, Artificial Sequence
  Primer
gctctaggttccgggtactaagcaagatgtggtctcctgtgtgta

SEQ ID NO: 40 - DNA, 45 bp, Artificial Sequence
  Primer
tcgaacgtgtcattggtactgccccacgataaaccaaaactcacc

SEQ ID NO: 41 - DNA, 45 bp, Artificial Sequence
  Primer
atagactagcctgccggtcaactctctccctctacccagctctga

SEQ ID NO: 42 - DNA, 45 bp, Artificial Sequence
  Primer
aatatcgtaagacatccgcggaatcgaagtactgatacggggagc

SEQ ID NO: 43 - DNA, 45 bp, Artificial Sequence
  Primer
aacagtctaacctacgcgagtctggatcggccaagcacccgggag

SEQ ID NO: 44 - DNA, 45 bp, Artificial Sequence
  Primer
atacgtcttaccgcacataggctcctagaaatgctctgctgctcc

SEQ ID NO: 45 - DNA, 45 bp, Artificial Sequence
  Primer
gtaacctattcgtgactagcaccacctcccaggaaacagttctga

SEQ ID NO: 46 - DNA, 45 bp, Artificial Sequence
  Primer
gagtatcttacctggtctaggagaaagctggcctctttggggagt

SEQ ID NO: 47 - DNA, 45 bp, Artificial Sequence
  Primer
gtatctaattcgtgagtcggaacatttaggcatatcactgttttt

SEQ ID NO: 48 - DNA, 45 bp, Artificial Sequence
  Primer
gtactacattcgtgcgatggagattcaaaaaacagtaggcagagt

SEQ ID NO: 49 - DNA, 45 bp, Artificial Sequence
  Primer
atgtatccgaagtcgtagtggttcagctgggtgactctgcaccag

SEQ ID NO: 50 - DNA, 45 bp, Artificial Sequence
  Primer
atttgacgaacgtatgccgccgggggagtccagcgttgacagagc

SEQ ID NO: 51 - DNA, 45 bp, Artificial Sequence
  Primer
aaattcgccacctagatcgagctcaccaatggttccacgtgttca

SEQ ID NO: 52 - DNA, 45 bp, Artificial Sequence
  Primer
aattatcggaactcgtcgctgaaggttggcaggccagggacaaca

SEQ ID NO: 53 - DNA, 45 bp, Artificial Sequence
  Primer
tatttacgaaccttgggagcctttccaagatctttcttgacaaac

SEQ ID NO: 54 - DNA, 45 bp, Artificial Sequence
  Primer
agcgactgtaaactaatcgggtttatgtctctgagcgagcagaga

SEQ ID NO: 55 - DNA, 45 bp, Artificial Sequence
  Primer
ggaacttattatagagccggctgtctaaaggcaggcaggggtgag

SEQ ID NO: 56 - DNA, 45 bp, Artificial Sequence
  Primer
tccaggtctttaacgacgtgggtcactgagtgctgcttcctaaag

SEQ ID NO: 57 - DNA, 45 bp, Artificial Sequence
  Primer
tcgagtcctttaagatcgctactattttcccaatgggtctgagtt

SEQ ID NO: 58 - DNA, 45 bp, Artificial Sequence
  Primer
tcgatacgtttaatctccggcaaagcccctcctttcactctgtgt

SEQ ID NO: 59 - DNA, 45 bp, Artificial Sequence
  Primer
ccatccgattaaataccgtgggagcccccctgccctgtagctctc

SEQ ID NO: 60 - DNA, 45 bp, Artificial Sequence
  Primer
gattacgttaagttacggcgccaagaaagccctgcccagctcttt

SEQ ID NO: 61 - DNA, 45 bp, Artificial Sequence
  Primer
gttgaccgttagttatgcgattcctctgttatgttcatacattag

SEQ ID NO: 62 - DNA, 45 bp, Artificial Sequence
  Primer
ggttcgcttacgttgcatagaactggaagcattgagggcttctgg

SEQ ID NO: 63 - DNA, 45 bp, Artificial Sequence
  Primer
cgtatcgcttaacctctatgggcaggaaagccggtttccagagtc

SEQ ID NO: 64 - DNA, 45 bp, Artificial Sequence
  Primer
cgtacagcttacctactatgccttctggtcatacacattcattta

SEQ ID NO: 65 - DNA, 45 bp, Artificial Sequence
  Primer
cgtgcaagttaccgagctgatgatggctgctgagtttactgaggt

SEQ ID NO: 66 - DNA, 45 bp, Artificial Sequence
  Primer
cgtcgcgttagacagctcatggtggtcagtaaaagagataaagga

SEQ ID NO: 67 - DNA, 45 bp, Artificial Sequence
  Primer
tcgtcacgtttaggactatgtccatgggttgttttccaaacagtg

SEQ ID NO: 68 - DNA, 45 bp, Artificial Sequence
  Primer
tcgaagcgtttagaccatgttggcacatctggagaatgaagattg

SEQ ID NO: 69 - DNA, 45 bp, Artificial Sequence
  Primer
tcggacgctttagatgacttcaaaaacaatgtcttcctgttcccc

SEQ ID NO: 70 - DNA, 45 bp, Artificial Sequence
  Primer
cgtctagcttaatacctctgtgcctggcctggccttaggtgccac

SEQ ID NO: 71 - DNA, 45 bp, Artificial Sequence
  Primer
cgtctcagttaatagtacgggattcaataaaataacatggctaaa

SEQ ID NO: 72 - DNA, 45 bp, Artificial Sequence
  Primer
gcaatccgttatgtaaagggaacacgcacgacgacagcaggaaca

SEQ ID NO: 73 - DNA, 45 bp, Artificial Sequence
  Primer
tgaccacgtttcagaagctggaggaaatcaacgagatatattagc

SEQ ID NO: 74 - DNA, 45 bp, Artificial Sequence
  Primer
ccatatacttacacagcaggaaatctgtgccatgaagtcgcactt

SEQ ID NO: 75 - DNA, 45 bp, Artificial Sequence
  Primer
ctatcacgttagatccactgctgtcatggcctctccctggactgc

SEQ ID NO: 76 - DNA, 45 bp, Artificial Sequence
  Primer
cgtcacgttacctacatgataaaggcagaggcaaggtcctgtttg

SEQ ID NO: 77 - DNA, 45 bp, Artificial Sequence
  Primer
cgatccgattacaggccgatcagaggcaaggtcctgtttggagga

SEQ ID NO: 78 - DNA, 45 bp, Artificial Sequence
  Primer
ccatcggattacacacgagttgaggaaagggccgctttgcttttg

SEQ ID NO: 79 - DNA, 45 bp, Artificial Sequence
  Primer
cctgcacgttagaacactggtggggacaaacacccgcatgcacac

SEQ ID NO: 80 - DNA, 45 bp, Artificial Sequence
  Primer
ctcgcggcttagatcagctttggcaacggtggaagaggcctagaa

SEQ ID NO: 81 - DNA, 45 bp, Artificial Sequence
  Primer
ccctcgctggagatcgaatatggaaggagaatagtggaggggtgc

SEQ ID NO: 82 - DNA, 45 bp, Artificial Sequence
  Primer
cgcccagcttagagcgaattctccacgagtgactgtggggaacag

SEQ ID NO: 83 - DNA, 45 bp, Artificial Sequence
  Primer
gcggcgcgttcgacataattctgcaagcgaccccgaccaatctac

SEQ ID NO: 84 - DNA, 45 bp, Artificial Sequence
  Primer
gccgacgcttcgacagaattacaggcctcccaggagctcacactc

SEQ ID NO: 85 - DNA, 45 bp, Artificial Sequence
  Primer
gtaggcgattctagccaattacaagatctacatcgtgatgaacta

SEQ ID NO: 86 - DNA, 45 bp, Artificial Sequence
  Primer
gcacgtcgtattaggtagtcaggacacttaaatccacagagtcac

SEQ ID NO: 87 - DNA, 45 bp, Artificial Sequence
  Primer
actctcgacctagcgtaaggtctgaccttcagggtccaactacag

SEQ ID NO: 88 - DNA, 42 bp, Artificial Sequence
  Primer
atccacgatcctagagtcggtgcctactctcccaacccaaaa

SEQ ID NO: 89 - DNA, 42 bp, Artificial Sequence
  Primer
atccatagtcctaagtccggtccgcacagccggtcataaagc

SEQ ID NO: 90 - DNA, 45 bp, Artificial Sequence
  Primer
acgcggtcactcagcatataactcagctcacgcattattatgtta

SEQ ID NO: 91 - DNA, 45 bp, Artificial Sequence
  Primer
gtcgttgcacctagttgataatctgtgatattctctgtcttagac

SEQ ID NO: 92 - DNA, 45 bp, Artificial Sequence
  Primer
gtcgccgattctagttatgggctgttcctggactgtctgacctag

SEQ ID NO: 93 - DNA, 45 bp, Artificial Sequence
  Primer
cgtcggattagaccggatcaaattaagtgggtgaacaatgtgacc

SEQ ID NO: 94 - DNA, 45 bp, Artificial Sequence
  Primer
cccggacatggacgttaagtgagatcagtcctaccatgcacctac

SEQ ID NO: 95 - DNA, 24 bp, Artificial Sequence
  Primer
tgtttatccatgccataaattttg

SEQ ID NO: 96 - DNA, 18 bp, Artificial Sequence
  Primer
acggaccctgagcacaga

SEQ ID NO: 97 - DNA, 24 bp, Artificial Sequence
  Primer
ttttgaaagataagggaaagcaca

SEQ ID NO: 98 - DNA, 25 bp, Artificial Sequence
  Primer
taatacctagtcaccaacagtgacc

SEQ ID NO: 99 - DNA, 25 bp, Artificial Sequence
  Primer
ttcatgcgtattttaacacataatg

SEQ ID NO: 100 - DNA, 19 bp, Artificial Sequence
  Primer
tgggctgtaggggcaatat

SEQ ID NO: 101 - DNA, 20 bp, Artificial Sequence
  Primer
caaggacactgggaatcttg

SEQ ID NO: 102 - DNA, 27 bp, Artificial Sequence
  Primer
atttgcgttctacacattcatagtgtt

SEQ ID NO: 103 - DNA, 26 bp, Artificial Sequence
  Primer
tatgctaaagataactaaggcaaggc

SEQ ID NO: 104 - DNA, 21 bp, Artificial Sequence
  Primer
ggggtttcattgtaggtgaac

SEQ ID NO: 105 - DNA, 21 bp, Artificial Sequence
  Primer
tctgagagcagtcgacaggag

SEQ ID NO: 106 - DNA, 20 bp, Artificial Sequence
  Primer
gaggacagcactgctgagtg

SEQ ID NO: 107 - DNA, 24 bp, Artificial Sequence
  Primer
accctacagtccttacctttccaa

SEQ ID NO: 108 - DNA, 21 bp, Artificial Sequence
  Primer
catggtcaaggtctgcattcc

SEQ ID NO: 109 - DNA, 18 bp, Artificial Sequence
  Primer
agctggctgagatcgagg

SEQ ID NO: 110 - DNA, 20 bp, Artificial Sequence
  Primer
cagcagacagacacaggtcc

SEQ ID NO: 111 - DNA, 25 bp, Artificial Sequence
  Primer
aaaaatatgactttttttttccccc

SEQ ID NO: 112 - DNA, 22 bp, Artificial Sequence
  Primer
tatacacaatgcctgcctgaca

SEQ ID NO: 113 - DNA, 27 bp, Artificial Sequence
  Primer
ccactccacaataatcagattttacac

SEQ ID NO: 114 - DNA, 20 bp, Artificial Sequence
  Primer
tttttcacacatggagggtg

SEQ ID NO: 115 - DNA, 19 bp, Artificial Sequence
  Primer
aggcgatgcagcagagaat

SEQ ID NO: 116 - DNA, 31 bp, Artificial Sequence
  Primer
atattagcattattagctgtacctcacttgt

SEQ ID NO: 117 - DNA, 20 bp, Artificial Sequence
  Primer
tgttaatgttggtgttggca

SEQ ID NO: 118 - DNA, 24 bp, Artificial Sequence
  Primer
atgacatccaagacagtttcctgt

SEQ ID NO: 119 - DNA, 21 bp, Artificial Sequence
  Primer
tggtgttgaatggctgaattg

SEQ ID NO: 120 - DNA, 25 bp, Artificial Sequence
  Primer
actaggcagtattttatgagccagc

SEQ ID NO: 121 - DNA, 25 bp, Artificial Sequence
  Primer
aagtttggtttaacatctgactggc

SEQ ID NO: 122 - DNA, 21 bp, Artificial Sequence
  Primer
gggaaattcgagggatttttc

SEQ ID NO: 123 - DNA, 26 bp, Artificial Sequence
  Primer
tcctcaatttccttacagtagaacat

SEQ ID NO: 124 - DNA, 19 bp, Artificial Sequence
  Primer
ccttcccccaactacctgg

SEQ ID NO: 125 - DNA, 19 bp, Artificial Sequence
  Primer
tctctggcgctcaagacac

SEQ ID NO: 126 - DNA, 23 bp, Artificial Sequence
  Primer
aaagtgagtgacagtggtgctct

SEQ ID NO: 127 - DNA, 21 bp, Artificial Sequence
  Primer
atctcatgacctgtggcattg

SEQ ID NO: 128 - DNA, 24 bp, Artificial Sequence
  Primer
tcagactggctgtattaaatcgtt

SEQ ID NO: 129 - DNA, 26 bp, Artificial Sequence
  Primer
agaaacgaaaacagcaagagtaaata

SEQ ID NO: 130 - DNA, 22 bp, Artificial Sequence
  Primer
aagcagtgaattgctcaaacca

SEQ ID NO: 131 - DNA, 31 bp, Artificial Sequence
  Primer
tgcatttcacataagtgcataattaatacta

SEQ ID NO: 132 - DNA, 26 bp, Artificial Sequence
  Primer
atgttttacaacaagctgtgtctctg

SEQ ID NO: 133 - DNA, 26 bp, Artificial Sequence
  Primer
actagactcaggactccatttacagc

SEQ ID NO: 134 - DNA, 23 bp, Artificial Sequence
  Primer
gaaacacaaaaaccacaggacaa

SEQ ID NO: 135 - DNA, 19 bp, Artificial Sequence
  Primer
ttgaacccaggtttccagc

SEQ ID NO: 136 - DNA, 21 bp, Artificial Sequence
  Primer
acgagtgccttctggaagcta

SEQ ID NO: 137 - DNA, 19 bp, Artificial Sequence
  Primer
actgtgcagccagagatgg

SEQ ID NO: 138 - DNA, 22 bp, Artificial Sequence
  Primer
aggaggagctcagagttggact

SEQ ID NO: 139 - DNA, 25 bp, Artificial Sequence
  Primer
aatctatattatcacccttccccac

SEQ ID NO: 140 - DNA, 27 bp, Artificial Sequence
  Primer
cagacagagaaatagctacaaaacagc

SEQ ID NO: 141 - DNA, 19 bp, Artificial Sequence
  Primer
atggcagaggctgtgtgtg

SEQ ID NO: 142 - DNA, 21 bp, Artificial Sequence
  Primer
ttaagggggcctaaaaagctg

SEQ ID NO: 143 - DNA, 22 bp, Artificial Sequence
  Primer
atcttcctcactgccctacttg

SEQ ID NO: 144 - DNA, 26 bp, Artificial Sequence
  Primer
cccagtaagagaaatcatacgagaag

SEQ ID NO: 145 - DNA, 19 bp, Artificial Sequence
  Primer
tnngccttggctctcagcc

SEQ ID NO: 146 - DNA, 28 bp, Artificial Sequence
  Primer
taattttggttgctatagattccaagtc

SEQ ID NO: 147 - DNA, 27 bp, Artificial Sequence
  Primer
ttagacacatgcttagaagaagatgct

SEQ ID NO: 148 - DNA, 23 bp, Artificial Sequence
  Primer
ataagtggtctccctgcttatgg

SEQ ID NO: 149 - DNA, 27 bp, Artificial Sequence
  Primer
aaaggaataaggtcaagacttacatcc

SEQ ID NO: 150 - DNA, 18 bp, Artificial Sequence
  Primer
tgcgcccctgcccttttc

SEQ ID NO: 151 - DNA, 25 bp, Artificial Sequence
  Primer
tatatcttatgtgcttttgaacggc

SEQ ID NO: 152 - DNA, 23 bp, Artificial Sequence
  Primer
ccttcctctgaatgatcaggtct

SEQ ID NO: 153 - DNA, 21 bp, Artificial Sequence
  Primer
ttttgaggtttctggggaagg

SEQ ID NO: 154 - DNA, 21 bp, Artificial Sequence
  Primer
aatctcagaatttccaagccg

SEQ ID NO: 155 - DNA, 28 bp, Artificial Sequence
  Primer
tggcacaagtaaaaactccataaatatt

SEQ ID NO: 156 - DNA, 21 bp, Artificial Sequence
  Primer
taggataaattgcctgccatg

SEQ ID NO: 157 - DNA, 19 bp, Artificial Sequence
  Primer
ttcaaccccaaaaggcaaa

SEQ ID NO: 158 - DNA, 22 bp, Artificial Sequence
  Primer
aggggaatgtaattacggaggc

SEQ ID NO: 159 - DNA, 18 bp, Artificial Sequence
  Primer
ctgtcccagaggcccttg

SEQ ID NO: 160 - DNA, 21 bp, Artificial Sequence
  Primer
tggcttatgacattcgcattt

SEQ ID NO: 161 - DNA, 24 bp, Artificial Sequence
  Primer
aacttttcatgacagagacaggga

SEQ ID NO: 162 - DNA, 24 bp, Artificial Sequence
  Primer
tggtctcttctgggagtgatctaa

SEQ ID NO: 163 - DNA, 27 bp, Artificial Sequence
  Primer
aaaaggaaagtcatcctgagtcttact

SEQ ID NO: 164 - DNA, 34 bp, Artificial Sequence
  Primer
ttttaatatacggtagtgacattctagtagatga

SEQ ID NO: 165 - DNA, 29 bp, Artificial Sequence
  Primer
aagaagaaagtatccaaaacctagacaga

SEQ ID NO: 166 - DNA, 21 bp, Artificial Sequence
  Primer
tcacagcaaaaggaccagatg

SEQ ID NO: 167 - DNA, 23 bp, Artificial Sequence
  Primer
ttccttgttttaatggagacgct

SEQ ID NO: 168 - DNA, 20 bp, Artificial Sequence
  Primer
tgccaggcaaccacttagtt

SEQ ID NO: 169 - DNA, 20 bp, Artificial Sequence
  Primer
gcatgacagagaaggcactt

SEQ ID NO: 170 - DNA, 23 bp, Artificial Sequence
  Primer
atgaacttacagagcaagatcgc

SEQ ID NO: 171 - DNA, 29 bp, Artificial Sequence
  Primer
tttaaaatgttctttcaaagactaaacgc

SEQ ID NO: 172 - DNA, 20 bp, Artificial Sequence
  Primer
ttgttttgtcttgaggcacg

SEQ ID NO: 173 - DNA, 19 bp, Artificial Sequence
  Primer
atgggttgcttcacattgg

SEQ ID NO: 174 - DNA, 21 bp, Artificial Sequence
  Primer
gtgtacgtgcgtgcacatatg

SEQ ID NO: 175 - DNA, 23 bp, Artificial Sequence
  Primer
attcaatgctggactttttcaag

SEQ ID NO: 176 - DNA, 26 bp, Artificial Sequence
  Primer
tttggcttgcttttccatataactta

SEQ ID NO: 177 - DNA, 25 bp, Artificial Sequence
  Primer
acggtggagaatcttaggaatgtaa

SEQ ID NO: 178 - DNA, 23 bp, Artificial Sequence
  Primer
tgctgagtaaacccaaactctca

SEQ ID NO: 179 - DNA, 20 bp, Artificial Sequence
  Primer
tttgggaaactccaggtcag

SEQ ID NO: 180 - DNA, 20 bp, Artificial Sequence
  Primer
agtggctggaaagaggactg

SEQ ID NO: 181 - DNA, 23 bp, Artificial Sequence
  Primer
cactgccctagagacagagtttg

SEQ ID NO: 182 - DNA, 24 bp, Artificial Sequence
  Primer
aaaagaaactcaaggtgaacctga

SEQ ID NO: 183 - DNA, 18 bp, Artificial Sequence
  Primer
tccacggcttccccctta

SEQ ID NO: 184 - DNA, 19 bp, Artificial Sequence
  Primer
atcccaggacagggtcatg

SEQ ID NO: 185 - DNA, 21 bp, Artificial Sequence
  Primer
tcccttacccagatgtgagga

SEQ ID NO: 186 - DNA, 26 bp, Artificial Sequence
  Primer
ggaggtgagaacatagcagagataat

SEQ ID NO: 187 - DNA, 25 bp, Artificial Sequence
  Primer
taattcagtgagtgtgagtccttgg

SEQ ID NO: 188 - DNA, 20 bp, Artificial Sequence
  Primer
ggtctgtgcaaactccctca

SEQ ID NO: 189 - DNA, 19 bp, Artificial Sequence
  Primer
gcaggaacatttggcctgt

SEQ ID NO: 190 - DNA, 26 bp, Artificial Sequence
  Primer
tgaatccaagctcttaacttgctact

SEQ ID NO: 191 - DNA, 19 bp, Artificial Sequence
  Primer
caggaacaggaacgcaatg

SEQ ID NO: 192 - DNA, 23 bp, Artificial Sequence
  Primer
attgggtgtctcagaggcataat

SEQ ID NO: 193 - DNA, 32 bp, Artificial Sequence
  Primer
agtatgttaattagttatacaataccaagggg

SEQ ID NO: 194 - DNA, 19 bp, Artificial Sequence
  Primer
tcatcctgcactgtcaggc

SEQ ID NO: 195 - DNA, 23 bp, Artificial Sequence
  Primer
gcagtaactaggtttgcatctga

SEQ ID NO: 196 - DNA, 24 bp, Artificial Sequence
  Primer
taactaggtttgcatctgatggtg

SEQ ID NO: 197 - DNA, 24 bp, Artificial Sequence
  Primer
taacaaaaggatctcacacttggc

SEQ ID NO: 198 - DNA, 27 bp, Artificial Sequence
  Primer
aagaaatggaagcatatgactctaagc

SEQ ID NO: 199 - DNA, 19 bp, Artificial Sequence
  Primer
ccccgtagagtcaaagcac

SEQ ID NO: 200 - DNA, 26 bp, Artificial Sequence
  Primer
ctattttgtgttcatcttctgaaagc

SEQ ID NO: 201 - DNA, 30 bp, Artificial Sequence
  Primer
acatttgtagagaatgccctttatatatgt

SEQ ID NO: 202 - DNA, 20 bp, Artificial Sequence
  Primer
ttttagggcacgagacaagg

SEQ ID NO: 203 - DNA, 22 bp, Artificial Sequence
  Primer
tgacctcccaggttcaattagc

SEQ ID NO: 204 - DNA, 20 bp, Artificial Sequence
  Primer
ttaaagcctcatggctctgg

SEQ ID NO: 205 - DNA, 23 bp, Artificial Sequence
  Primer
tctctgagaccactcagcaactc

SEQ ID NO: 206 - DNA, 27 bp, Artificial Sequence
  Primer
atttagaaattgtagcaaacacgttgt

SEQ ID NO: 207 - DNA, 24 bp, Artificial Sequence
  Primer
aaactctcacatcagcatgacact

SEQ ID NO: 208 - DNA, 20 bp, Artificial Sequence
  Primer
aatgaaccaggcagggagat

SEQ ID NO: 209 - DNA, 18 bp, Artificial Sequence
  Primer
tggggtgtggagccaaga

SEQ ID NO: 210 - DNA, 28 bp, Artificial Sequence
  Primer
atttgtagttcttctgaaaccttcagtt

SEQ ID NO: 211 - DNA, 22 bp, Artificial Sequence
  Primer
caaaccctctaggctttcattg

SEQ ID NO: 212 - DNA, 25 bp, Artificial Sequence
  Primer
atgaaacctcataaaaggaacgact

SEQ ID NO: 213 - DNA, 22 bp, Artificial Sequence
  Primer
ttcagcccatgtagacttggtt

SEQ ID NO: 214 - DNA, 24 bp, Artificial Sequence
  Primer
atcaggaacagagtggttactgca

SEQ ID NO: 215 - DNA, 18 bp, Artificial Sequence
  Primer
acttagttggggccaggc

SEQ ID NO: 216 - DNA, 19 bp, Artificial Sequence
  Primer
agaaggactggctgggatg

SEQ ID NO: 217 - DNA, 24 bp, Artificial Sequence
  Primer
aatcgttttgctcgttctactttc

SEQ ID NO: 218 - DNA, 18 bp, Artificial Sequence
  Primer
tttgccaaacggcatttc

SEQ ID NO: 219 - DNA, 22 bp, Artificial Sequence
  Primer
aaacgaagtctccagtgagacg

SEQ ID NO: 220 - DNA, 19 bp, Artificial Sequence
  Primer
tccccatccaattcactgg

SEQ ID NO: 221 - DNA, 23 bp, Artificial Sequence
  Primer
gtttcactaagaggcagcgaatc

SEQ ID NO: 222 - DNA, 32 bp, Artificial Sequence
  Primer
tgagtatgttttctatctcttttgtctagaaa

SEQ ID NO: 223 - DNA, 18 bp, Artificial Sequence
  Primer
ccaacaacctctgggtgg

SEQ ID NO: 224 - DNA, 20 bp, Artificial Sequence
  Primer
ttagggcatccactgtcctg

SEQ ID NO: 225 - DNA, 26 bp, Artificial Sequence
  Primer
caagaacatataatgaacgaccttgg

SEQ ID NO: 226 - DNA, 22 bp, Artificial Sequence
  Primer
gcattttccaaatcaagctgaa

SEQ ID NO: 227 - DNA, 25 bp, Artificial Sequence
  Primer
aaaaataaaatcacaggtgctcagg

SEQ ID NO: 228 - DNA, 24 bp, Artificial Sequence
  Primer
acttaatatgcctgcctgtcattc

SEQ ID NO: 229 - DNA, 26 bp, Artificial Sequence
  Primer
acagtaagggagagtagcaagaaatc

SEQ ID NO: 230 - DNA, 19 bp, Artificial Sequence
  Primer
tgaatcaatggggttgggt

SEQ ID NO: 231 - DNA, 21 bp, Artificial Sequence
  Primer
taggtgaatcaatggggttgg

SEQ ID NO: 232 - DNA, 24 bp, Artificial Sequence
  Primer
tgacaagtaaacaagatttggcac

SEQ ID NO: 233 - DNA, 27 bp, Artificial Sequence
  Primer
agttgctcagcactgttttataatctg

SEQ ID NO: 234 - DNA, 22 bp, Artificial Sequence
  Primer
tgaggacgaatggttttctttc

SEQ ID NO: 235 - DNA, 20 bp, Artificial Sequence
  Primer
gatggagaagcgatgtttgc

SEQ ID NO: 236 - DNA, 24 bp, Artificial Sequence
  Primer
tggatgagcagtcagagagtctac

SEQ ID NO: 237 - DNA, 23 bp, Artificial Sequence
  Primer
aattcatcaccgatattcttggg

SEQ ID NO: 238 - DNA, 21 bp, Artificial Sequence
  Primer
atttggggtgaccaagtcatg

SEQ ID NO: 239 - DNA, 18 bp, Artificial Sequence
  Primer
accagtccccacacccac

SEQ ID NO: 240 - DNA, 19 bp, Artificial Sequence
  Primer
cctggggacagttcaaggg

SEQ ID NO: 241 - DNA, 19 bp, Artificial Sequence
  Primer
gacagttcaaggggcaaag

SEQ ID NO: 242 - DNA, 19 bp, Artificial Sequence
  Primer
aatgtgggaggcacaggac

SEQ ID NO: 243 - DNA, 22 bp, Artificial Sequence
  Primer
agaacctgttccacctaaaccc

SEQ ID NO: 244 - DNA, 22 bp, Artificial Sequence
  Primer
atccagagagagggcttcagag

SEQ ID NO: 245 - DNA, 19 bp, Artificial Sequence
  Primer
tggtggcagtggttggcta

SEQ ID NO: 246 - DNA, 23 bp, Artificial Sequence
  Primer
tagatatgtctgggcatcgagaa

SEQ ID NO: 247 - DNA, 22 bp, Artificial Sequence
  Primer
ttgtgaatcccatatccaggaa

SEQ ID NO: 248 - DNA, 26 bp, Artificial Sequence
  Primer
atgtcttggaaatcatcttttcttct

SEQ ID NO: 249 - DNA, 21 bp, Artificial Sequence
  Primer
agctggatcatcagggtcttc

SEQ ID NO: 250 - DNA, 21 bp, Artificial Sequence
  Primer
tgctgcataaattctgccaat

SEQ ID NO: 251 - DNA, 19 bp, Artificial Sequence
  Primer
acgcaggaaaaagccacag

SEQ ID NO: 252 - DNA, 22 bp, Artificial Sequence
  Primer
tctcacacagcctcagaagacc

SEQ ID NO: 253 - DNA, 20 bp, Artificial Sequence
  Primer
gcagcctggttcagagacaa

SEQ ID NO: 254 - DNA, 18 bp, Artificial Sequence
  Primer
ttgtgggtgcgccatcta

SEQ ID NO: 255 - DNA, 21 bp, Artificial Sequence
  Primer
aaggtgggaaaagtgaagcaa

SEQ ID NO: 256 - DNA, 20 bp, Artificial Sequence
  Primer
tggttgatgcccactcctag

SEQ ID NO: 257 - DNA, 20 bp, Artificial Sequence
  Primer
atgatgcccttcacttgagc

SEQ ID NO: 258 - DNA, 19 bp, Artificial Sequence
  Primer
agcatcagcacactcagcg

SEQ ID NO: 259 - DNA, 25 bp, Artificial Sequence
  Primer
attaggagacaatgacactgacgtt

SEQ ID NO: 260 - DNA, 19 bp, Artificial Sequence
  Primer
cgtgcacactctccagtgg

SEQ ID NO: 261 - DNA, 29 bp, Artificial Sequence
  Primer
ggctcaaagtaggttatctaaataaatgg

SEQ ID NO: 262 - DNA, 22 bp, Artificial Sequence
  Primer
atagcaacacttggactccgaa

SEQ ID NO: 263 - DNA, 19 bp, Artificial Sequence
  Primer
tttttctggcctgtgaggg

SEQ ID NO: 264 - DNA, 20 bp, Artificial Sequence
  Primer
ttagtgggacccctggctat

SEQ ID NO: 265 - DNA, 20 bp, Artificial Sequence
  Primer
agccacacttagtgggaccc

SEQ ID NO: 266 - DNA, 21 bp, Artificial Sequence
  Primer
tgttttcagaacctggagagg

SEQ ID NO: 267 - DNA, 20 bp, Artificial Sequence
  Primer
tcctcatggtgttctgtgca

SEQ ID NO: 268 - DNA, 22 bp, Artificial Sequence
  Primer
aaaacttgcctgtgatgtgtgg

SEQ ID NO: 269 - DNA, 19 bp, Artificial Sequence
  Primer
cccagcacacctgcatgta

SEQ ID NO: 270 - DNA, 24 bp, Artificial Sequence
  Primer
ataccatcattttcacagggaaac

SEQ ID NO: 271 - DNA, 29 bp, Artificial Sequence
  Primer
aaaatgtctagaatgaaatctgttctctg

SEQ ID NO: 272 - DNA, 19 bp, Artificial Sequence
  Primer
aaattcagcccagccatcc

SEQ ID NO: 273 - DNA, 18 bp, Artificial Sequence
  Primer
tgcaggagattgtggtgg

SEQ ID NO: 274 - DNA, 23 bp, Artificial Sequence
  Primer
accaatttttcttgaggttccct

SEQ ID NO: 275 - DNA, 20 bp, Artificial Sequence
  Primer
atgtgtcctcatggagaggc

SEQ ID NO: 276 - DNA, 27 bp, Artificial Sequence
  Primer
agcatagcgtggcttacttacttattt

SEQ ID NO: 277 - DNA, 18 bp, Artificial Sequence
  Primer
ggggaaggcaccgtcaca

SEQ ID NO: 278 - DNA, 21 bp, Artificial Sequence
  Primer
ttgaatccagagacacggaac

SEQ ID NO: 279 - DNA, 20 bp, Artificial Sequence
  Primer
atgtccacgttgcattctgc

SEQ ID NO: 280 - DNA, 18 bp, Artificial Sequence
  Primer
atcccgtcacttgccctg

SEQ ID NO: 281 - DNA, 18 bp, Artificial Sequence
  Primer
accgcaccctctgtggat

SEQ ID NO: 282 - DNA, 19 bp, Artificial Sequence
  Primer
agggtgctggcagtagagc